SEQUENCE LISTING (18 sequences)

SEQ ID NO: 1 - protein, 4 aa, Artificial Sequence
  peptide 1
RPRL

SEQ ID NO: 2 - protein, 4 aa, Artificial Sequence
  Peptide 2
QPRL

SEQ ID NO: 3 - protein, 14 aa, Artificial Sequence
  peptide 3
QRRCMPLHSRVPFP

SEQ ID NO: 4 - protein, 14 aa, Artificial Sequence
  Peptide 4
QRRCMPLHRSVPFP

SEQ ID NO: 5 - protein, 13 aa, Artificial Sequence
  Peptide 5
QRPRLSHKGPMPA

SEQ ID NO: 6 - protein, 12 aa, Artificial Sequence
  Peptide 6
QRPRLSHKGPMP

SEQ ID NO: 7 - protein, 13 aa, Artificial Sequence
  Peptide 7
QRPRLSHKGPMPF

SEQ ID NO: 8 - protein, 12 aa, Artificial Sequence
  Peptide 8
RPRLSHKGPMPF

SEQ ID NO: 9 - protein, 17 aa, Artificial Sequence
  Peptide 9
SEQCRPRLCKHCRPRLC

SEQ ID NO: 10 - protein, 13 aa, Artificial Sequence
  Peptide 10
QPRLSHKGPMPDF

SEQ ID NO: 11 - protein, 32 aa, Artificial Sequence
  Peptide 11
QRPVNLTMRRKLRKHNCLQRRCMPLHSRVPFP

SEQ ID NO: 12 - protein, 32 aa, Artificial Sequence; identical to SEQ ID NO: 11
  Peptide 12

SEQ ID NO: 13 - protein, 31 aa, Artificial Sequence
  Peptide 13
RPVNLTMRRKLRKHNCLQRRCMPLHRSVPFP

SEQ ID NO: 14 - protein, 36 aa, Artificial Sequence
  Peptide 14
LVQPRGSRNGPGPWQGGRRKFRRQRPRLSHKGPMPF

SEQ ID NO: 15 - protein, 17 aa, Artificial Sequence
  Peptide 15
KFRRQRPRLSHKGPMPF

SEQ ID NO: 16 - protein, 16 aa, Artificial Sequence
  Peptide 16
FRRQRPRLSHKGPMPF

SEQ ID NO: 17 - protein, 12 aa, Artificial Sequence
  Peptide 17
RPRLSHKGPMPF

SEQ ID NO: 18 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

(19) United States
(12) Patent Application Publication
(10) Pub. No.: US 2023/0226231 A1
(43) Pub. Date: Jul. 20, 2023

(54) CONJUGATED AND LABELLED APELIN, PREPARATION AND USES THEREOF

(71) Applicants: UNIVERSITE D'AIX MARSEILLE, MARSEILLE CEDEX 07 (FR); INSERM (INSTITUT NATIONAL DE LA SANTE ET DE LA RECHERCHE MEDICALE), PARIS (FR); ASSISTANCE PUBLIQUE HÔPITAUX DE MARSEILLE, MARSEILLE CEDEX 5 (FR)

(72) Inventors: BENJAMIN GUILLET, AIX EN PROVENCE (FR); AHLEM BOUHLEL, MARSEILLE (FR); ANAÏS MOYON, MIMET (FR); BÉATRICE MARIE FLORENCE LOUIS, MARSEILLE (FR); FRANÇOISE DIGNAT-GEORGE, MARSEILLE (FR); LAURE BALASSE, MARSEILLE (FR); PHILIPPE GARRIGUE, MARSEILLE (FR); SAMANTHA FERNANDEZ, MARIGNANE (FR)

(21) Appl. No.: 18/010,498

(22) PCT Filed: Jun. 18, 2021
(86) PCT No.: PCT/EP2021/066707

§ 371 Date: Dec. 15, 2022

(30) Foreign Application Priority Data
Jun. 18, 2020 (EP) 20305665.0

(51) Int. Cl. A61K 51/08; A61K 49/00; A61P 35/00

(52) U.S. Cl. CPC A61K 51/088; A61K 49/0004; A61P 35/00

(57) ABSTRACT

The invention relates to the field of imaging, diagnostic, internal vectorized radiotherapy and nuclear medicine. Inventors herein describe new products for use for labelling, detecting and/or imaging angiogenesis, vasculogenesis or a tissue or organ expressing the APJ receptor; for use for detecting, measuring, diagnosing, staging and/or monitoring angiogenesis, vasculogenesis, an angiogenesis- and/or vasculogenesis-related disease or disorder, and/or a disease or disorder inducing or modulating the expression of a APJ receptor in a tissue or organ; for use for preventing or treating angiogenesis, vasculogenesis, an angiogenesis- and/or vasculogenesis-related disease or disorder, and/or a disease or disorder inducing or modulating the expression of a APJ receptor in a tissue or organ; or for use for evaluating or monitoring the therapeutic effect of an angiogenic or antiangiogenic treatment or of an APJ receptor-targeted treatment. Compositions and kits comprising such products are also herein described as well as uses thereof.

The invention relates to the field of molecular imaging, diagnostic, internal vectorized radiotherapy and nuclear medicine. Inventors herein describe new products for use for labelling, detecting and/or imaging angiogenesis, vasculogenesis or a tissue or organ expressing the APJ receptor; for use for detecting, measuring, diagnosing, staging and/or monitoring angiogenesis, vasculogenesis, an angiogenesis- and/or vasculogenesis-related disease or disorder, and/or a disease or disorder inducing or modulating the expression of a APJ receptor in a tissue or organ; for use for preventing or treating angiogenesis, vasculogenesis, an angiogenesis- and/or vasculogenesis-related disease or disorder, and/or a disease or disorder inducing or modulating the expression of a APJ receptor in a tissue or organ; or for use for evaluating or monitoring the therapeutic effect of an angiogenic or antiangiogenic treatment or of an APJ receptor-targeted treatment. Compositions and kits comprising such products are also herein described as well as uses thereof.

### BACKGROUND

Positron emission tomography (“PET” or “PET scan”) or Positron Emission Tomography-Computed Tomography (PET-CT) is a nuclear medicine imaging technique that is used to observe molecular or metabolic processes in the body. PET is based on the general principle of scintigraphy which consists in introducing into the body a radiotracer whose in vivo behaviors allow to characterize by imaging the functioning of an organ or the tissue expression of a molecular target. This radiotracer is introduced into the body on a biologically active molecule and is marked by a radioactive element which emits positrons whose annihilation produces two photons. It is the detection in coincidence of these photons that allows the location of the place of their emission and therefore the concentration of the tracer at each point of the targeted tissue, typically of the organ. Three-dimensional images showing in color the zones of high concentration of the tracer within the body are then constructed by computer analysis. In modern PET-CT scanners, three dimensional imaging is often accomplished with the aid of a CT X-ray scan performed on the patient during the same session, in the same machine.

Thus, PET makes it possible to visualize the activities of the cell metabolism: functional imaging as opposed to so-called structural imaging techniques such as those based on X-rays (radiology or CT-scanner) that produce images of anatomy. Therefore, positron emission tomography (PET) is a diagnostic tool that detects certain pathologies that result in an alteration of normal physiology such as cancers, but also dementia for example. The expression “molecular imaging” is more and more used, since the tracers make it possible to produce images of molecular targets: targeting a particular receptor, marking the deposition of amyloid plaques, acquiring images of hypoxic processes, hormone receptors, etc.

Single-photon emission computed tomography (“SPECT” or “SPECT-CT”, or less commonly, “SPET” or “SPET-CT”) is a nuclear medicine tomographic imaging technique. It is very similar to conventional nuclear medicine planar imaging using a gamma camera. However, it is able to provide true 3D information. This information is typically presented as cross-sectional slices through the patient but can be freely reformatted or manipulated as required.

The technique requires delivery of a gamma-emitting radioisotope (a radionuclide) into the patient, normally through injection into the bloodstream. On occasion, the radioisotope is a simple soluble dissolved ion, such as an isotope of gallium (III). Most of the time, though, a radioisotope marker is attached to a specific ligand to create a radioligand also called “radiopharmaceutical” for clinical applications, whose properties bind it to a place of interest in the body where the ligand concentration is seen by a gamma camera.

SPECT is similar to PET in its use of radioactive tracer material and detection of gamma rays. In contrast with PET, however, the tracers used in SPECT emit gamma radiation that is measured directly, whereas PET tracers emit positrons that annihilate with electrons up to a few millimeters away, causing two gamma photons to be emitted in opposite directions. A PET scanner detects these emissions “coincidently” in time, which provides more radiation event localization information and, thus, higher spatial resolution images than SPECT (which has about 1 cm resolution). SPECT scans, however, are significantly less expensive than PET scans, in part because they are able to use more easily obtained longer-life radioisotopes than PET.

Nuclear medicine is a medical specialty involving the application of radioactive substances in the diagnosis and treatment of disease. Nuclear medicine, in a sense, is “radiology done inside out” or “endoradiology” because it records radiation emitting from the body rather than radiation that is generated by external sources like X-rays. Single Photon Emission Computed Tomography or SPECT and Positron Emission Tomography or PET scans are the two most common imaging modalities in nuclear medicine.

Evaluation of angiogenic/vasculogenic status of tissue using imaging is of crucial interest in the management of patients suffering of cardiovascular ischemic diseases to evaluate tissue regenerative status and/or pro-angiogenic therapy efficiency, and in the management of cancer patients to evaluate neoangiogenic status and/or anti-angiogenic therapy efficiency. This is in agreement with recent FDA incitation that promote companion tools development to identify patients likely to respond to therapeutic treatments and to selectively adapt treatments. Moreover, PET imaging represents a relevant companion tool thanks to its high sensitivity and quantitative assessment of molecular targets in whole body imaging applications.

Despite a long-felt need, there is to date no satisfactory tool for use for detecting and/or measuring vasculogenesis and/or angiogenesis. ⁶⁸Ga-NODAGA-THERANOST™ (“RGD”) is used to mark platelets and cancer tissue expressing αvβ3 integrin. However, αvβ3 integrin is not specific to angiogenesis/vasculogenesis and RGD-based imaging often associated with poor tissue targeting. ⁶⁸Ga-NODAGA-VEGF is used to mark receptors of vascular endothelial growth factor (VEGF), namely VEGFR1 and VEGFR2, but suffers from a lack of specificity as it cannot discriminate VEGFR2 from VEGFR1, whereas VEGFR2 only is associated with neoangiogenesis.

### BRIEF DESCRIPTION OF THE INVENTION

Inventors now herein describe a new product which is an Apelin or a functional fragment thereof, in particular a fragment comprising SEQ ID NO: 1, SEQ ID NO:2, SEQ ID NO: 3 or SEQ ID NO: 4, preferably the (F13A)Apelin isoform (also herein identified as “F13A” or “(F13A)Apelin”), wherein the Apelin or functional fragment thereof is labeled with a radioactive element, typically a pharmaceutically acceptable radioactive element. In a preferred embodiment, the radiolabeled Apelin or functional fragment thereof is in addition conjugated to a chelator.

In the context of the present description, the term “Apelin” designates any known Apelin or a functional fragment thereof, typically a fragment comprising SEQ ID NO: 1, SEQ ID NO:2, SEQ ID NO: 3 or SEQ ID NO: 4. The term “Apelin” preferably designates (F13A)Apelin (SEQ ID NO: 5), also herein identified as “F13A”.

Are in particular herein described [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, Al[¹⁸F]F-DOTAGA-Apelin, [⁶⁴Cu]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [¹⁷⁷Lu]Lu-DOTA-Apelin, [¹⁷⁷Lu]Lu-DOTAGA-Apelin, [¹⁷⁷Lu]Lu-DKFZ-Apelin, [¹⁷⁷Lu]Lu-AAZTA-Apelin, [²²⁵Ac]Ac-DOTA-Apelin, [Pb²¹²]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin and [¹¹¹In]In-DTPA-Apelin, [¹⁴⁹Tb]Tb-DOTA-Apelin, [¹⁴⁹Tb]Tb-DOTAGA-Apelin, [¹⁵²Tb]Tb-DOTA-Apelin, [¹⁵²Tb]Tb-DOTAGA-Apelin, [¹⁵⁵Tb]Tb-DOTA-Apelin, [¹⁵⁵Tb]Tb-DOTAGA-Apelin, [16 Tb]Tb-DOTA-Apelin and [¹⁶¹Tb]Tb-DOTAGA-Apelin. Each of these conjugated and labelled “Apelin” is typically used or for use as a Single Photon Emission Computed Tomography (SPECT-CT) tracer or as a Positron Emission Tomography-Computed Tomography (PET-CT) tracer.

Also herein described are [¹⁷⁷Lu]Lu-DOTA-Apelin, [¹⁷⁷Lu]Lu-DOTAGA-Apelin, [¹⁷⁷Lu]Lu-DKFZ-Apelin, [²²⁵Ac]Ac-DOTA-Apelin, [Pb²¹²]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin, [¹⁴⁹Tb]Tb-DOTA-Apelin, [¹⁴⁹Tb]Tb-DOTAGA-Apelin, [¹⁶¹Tb]Tb-DOTA-Apelin, or [¹⁶¹Tb]Tb-DOTAGA-Apelin. Each of these products is typically used or for use as a therapeutic agent, typically in nuclear medicine therapy.

Objects herein described further include compositions and kits comprising anyone of the herein described radiolabeled “Apelin” (also herein identified as “labeled Apelin”), typically (radio)labeled conjugated “Apelin”, or a combination thereof. The compositions and kits preferably comprise the radiolabeled “Apelin”, typically the (radio)labeled conjugated “Apelin” together with a pharmaceutically acceptable diluent, excipient, carrier or support.

Inventors in addition herein describe a radiolabeled Apelin, typically a (radio)labeled conjugated Apelin, and a composition comprising the same for use for, or for use in an in vitro, ex vivo or in vivo method of, labelling, detecting and/or imaging angiogenesis, vasculogenesis or a tissue or organ expressing the APJ receptor; of detecting, determining, evaluating, measuring, diagnosing, staging and/or monitoring angiogenesis, vasculogenesis, an angiogenesis- and/or vasculogenesis-related disease or disorder, and/or a disease or disorder inducing or modulating the expression of a APJ receptor in a tissue or organ; of determining the therapeutic eligibility of a subject to a therapeutic treatment involving a particular agent or protocol; of preventing or treating angiogenesis, vasculogenesis, an angiogenesis- and/or vasculogenesis-related disease or disorder, and/or a disease or disorder inducing or modulating the expression of a APJ receptor in a tissue or organ; or of evaluating or monitoring the therapeutic effect of an angiogenic or antiangiogenic treatment, or of an APJ receptor-targeted treatment, in a subject. Herein described in particular is a (radio)labeled Apelin, typically a (radio)labeled conjugated Apelin, and a composition comprising the same for use for, or for use in an in vitro, ex vivo or in vivo method of labelling, detecting and/or imaging an APJ receptor-expressing tissue in a subject, typically a hypoxic tissue, for example a post-ischemic tissue or a tumor, in particular a cancerous tumor, the APJ receptor-expressing tissue being typically a APJ receptor-overexpressing tissue.

Also herein described are a (radio)labeled Apelin, typically a (radio)labeled conjugated Apelin, and a composition comprising the same for use for, or for use in a method of, preventing or treating, in a subject, angiogenesis, vasculogenesis, an angiogenesis- and/or vasculogenesis-related disease or disorder, and/or a disease or disorder inducing or modulating the expression of a APJ receptor in a tissue or organ, in particular a disease or disorder associated to a tissue expressing an APJ receptor, typically a hypoxic tissue, for example a post-ischemic tissue or a tumor, in particular a cancerous tumor, the APJ receptor-expressing tissue being typically a APJ receptor-overexpressing tissue.

Compositions and kits comprising such products are also herein described as well as uses thereof.

A typical kit of the invention comprises an Apelin (typically an Apelin comprising SE ID NO: 1), a radioactive element and preferably a chelator, in two or three distinct containers, or an Apelin-chelator conjugate in a single container and the radioactive element in a distinct container.

Further herein described is the use of such a kit for producing a (radio)labeled Apelin, typically a (radio)labeled conjugated Apelin.

### DETAILED DESCRIPTION OF THE INVENTION

Apelin/APJ has been involved in the lowering of blood pressure (Yeganeh-Hajahmadi M et al., 2017), the promotion of the adhesion of monocytes to human umbilical vein ECs (Li X et al., 2010; Liu M et al., 2018), and the enhancement of angiogenesis and vasodilation (Li Y et al., 2018). Eyries et al. (2008) demonstrated that hypoxia promoted apelin expression, which enhanced ECs proliferation and regenerative angiogenesis. In a mouse model of oxygen-induced retinopathy, the expression of apelin was increased during hypoxia and was significantly higher than the expression of VEGF (Kasai A et al., 2010; Kasai A et al., 2013). Apelin/APJ axis also regulates the cardiovascular system, fluid homeostasis, metabolic pathways, and angiogenesis through different signaling pathways and ECs polarization (Kasai A et al., 2010).

Apelin is involved in the pathophysiology of human heart failure. Foldes et al. demonstrated in 2003 higher expression levels of apelin mRNA in failing human hearts compared to normal tissue. Apelin increases cardiac output and lowers blood pressure and peripheral vascular resistance in patients with heart failure (Japp A G et al., 2010). Moreover, [Pyr¹]-apelin-13 injection into a rat model of myocardial infarction resulted in decreased infarct size, and increased heart rate and serum nitric oxide level for 7 consecutive days, indicating that apelin has a sustained cardioprotective effect against myocardial infarction (Azizi Y et al., 2013). Foussal et al. (2010) demonstrated also that apelin can abolish reactive oxygen species (ROS) formation, reduce oxidative stress and prevent cardiac hypertrophy. Wang et al. (2013) showed in apelin-knockout mice an increased myocardial infarction mortality, infarct size, and inflammation, with a reduction of the pro-survival pathway via phosphatidyl inositol 3-kinase/protein kinase B (PI3K/Akt) confirming the involvement of Apelin in myocardial infarction physiopathology.

As summarized in tables 1 and 2 of Wysocka M B et al. (2018), Apelin/APJ system is involved in many cardiovascular diseases and endothelial dysfunction related disease such as for example atherosclerosis, diabetes, obesity and brain ischemia.

Beyond cardiovascular pathologies, APJ/apelin is also involved in human cancers. An overexpression of Apelin was found in glioblastoma multiforme (Harford-Wright E et al., 2018), colon adenocarcinoma (Chen et al., Oncotarget 2017), non-small-cell lung cancer (Berta J et al., 2010), oral squamous cell carcinoma (Heo K et al., 2012), prostate cancer (Wan Y et al., 2015), and hepatocellular carcinoma. (Muto J et al., 2014). High levels of apelin expression are associated with poor prognosis in numerous cancer types (Berta J et al., 2010; Heo K et al., 2012; Wan Y et al., 2015). Hypoxia, a major feature of solid tumors, can promote malignant progression by enhancing the invasive and metastatic potential of cancer cells and can trigger tumor angiogenesis by stimulating the secretion of proangiogenic factors such as vascular endothelial growth factor (VEGF) (Cesirio J M S et al., 2017). Hypoxia is described as associated to APJ/apelin expression by Hou J et al. (2017).

In addition, many agonists and antagonists of APJ receptors have been discovered and synthetized and have shown therapeutic effects in animal models and patients. A lot of APJ agonists such as E339-3D6, ML233, MM07 and CMF-019 were discovered and synthetized one after another (Iturrioz X et al., 2010; Brame A L et al., 2015; Khan P et al., 2010; Trifonov L et al., 2018). At the same time, APJ antagonists were discovered. Apelin-13(F13A), a natural antagonist isoform of APJ, was found by Lee et al. (2005). Later, many other antagonists were discovered such as MM54, ML221 and puerarin (Macaluso N J M et al., 2011; Le Gonidec S et al., 2017; Maloney P R et al., 2012). A clinical trial to observe the serum apelin expression in cancer patients before and after bevacizumab treatment is active under the direction of the members of the Anti-Angiogenesis Biomarker Conference at Osaka University (Institutional Review Board authorization no 11331-2).

Inventors herein reveal that the complex between Apelin and its receptor (“APJ”) can be used as a valuable biomarker.

Herein described for the first time is a Apelin (herein generally identified as “Apelin”) labeled with a radioactive element (also herein identified as “labeled Apelin”, “radiolabeled Apelin” or “radiopeptide”), typically a pharmaceutically acceptable radioactive element. In a preferred embodiment, the labelled Apelin of the invention is conjugated to a chelator (and typically herein identified as a “(radio)labeled conjugated Apelin” or “conjugated and labeled Apelin” or “conjugated and radiolabeled Apelin”.

The preproprotein of apelin contains 77 amino acids, which can be enzymatically hydrolyzed into six active biological fragments, each comprising SEQ ID NO:1 (RPRL), SEQ ID NO:2 (QPRL), SEQ ID NO: 3 (QRRCMPLHSRVPFP) or SEQ ID NO: 4 (QRRCMPLHRSVPFP), respectively named apelin-36 (SEQ ID NO: 14), apelin-17 (SEQ ID NO: 15), apelin-16 (SEQ ID NO: 16), apelin-13 [SEQ ID NO: 7 (QRPRLSHKGPMPF)], apelin-12 (SEQ ID NO: 17), and the pyroglutamate modified form of apelin-13 ([Pyr1]-apelin-13 (Dray et al., 2015) [SEQ ID NO: 8 (Pyr-RPRLSHKGPMPF)].

The signal peptide in its amino-terminal (N-terminal) sequence directs apelin in the secretory pathway. Among these isoforms, apelin-13 is the most biologically active and most commonly used isoform [SEQ ID NO: 7 (QRPRLSHKGPMPF)] (Carroll A O et al., 1998; Wysocka M B et al., 2018).

[Pyr-1]-apelin-13 (SEQ ID NO: 8) represents the most common fragment in heart and brain whereas apelin-36 predominates in lung, testis and uterus while both fragments are prevalent in mammary gland (Kawamata Y et al., 2001; Maguire J J et al., 2009). Moreover, apelin fragments have also been found in plasma where apelin-17 and [pyr-1]-apelin-13 may represent the predominant forms (Mota N De et al., 2004). Apelin/APJ system is abundantly distributed in various tissues and cells of the human body. To date, studies have demonstrated that apelin can be detected in the right atrium, left ventricle, brain, lung, liver, and adrenal, and is especially highly expressed on endothelial cells (ECs) and smooth muscle cells (Kleinz M J et al., 2004; Li F Y et al., 2008).

In the context of the present description, the term “Apelin” designates any known Apelin protein (amino acid sequence) or a functional fragment thereof, i.e. a fragment which specifically recognizes and binds APJ, typically a fragment comprising SEQ ID NO: 1, SEQ ID NO: 2, SEQ ID NO: 3 or SEQID NO: 4. In a particular aspect herein described, the term “Apelin” designates “apelin-13” (“(F13A)Apelin”).

A Apelin, in particular a (F13A)Apelin, conjugated to a chelator and labeled with a radioactive element is herein described for the first time. In a typical aspect, the Apelin amino acid sequence comprises, or consists in, SEQ ID NO: 1, SEQ ID NO: 2, SEQ ID NO: 3 or SEQID NO: 4. In a particular aspect, the Apelin amino acid sequence comprises, or consists in, SEQ ID NO:5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

Inventors developed in particular a Gallium-68 (⁶⁸Ga) radiolabeled tracer combining an Apelin, typically an Apelin corresponding to an amino acid sequence comprising or consisting in SEQ ID NO: 1, SEQ ID NO: 2, SEQ ID NO: 3 or SEQ ID NO: 4, for example comprising anyone of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13, and a bifunctional chelator (NODAGA) which they tested in the context of a method of evaluating angiogenesis by imaging PET-CT (PET-SCAN) and validated in vivo, ex vivo and in vitro.

They have been able to produce in particular a (F13A)Apelin-NODAGA-⁶⁸Ga tracer with a radiochemical purity greater than 95% and a stability in serum for up to 2 hours. Rapid urinary and hepatic elimination after intravenous (IV) injection being observed, they concluded that such a tracer can be used in vivo in particular for imaging, typically for PET-CT and SPECT-CT imaging. The maintenance of the functional integrity of Apelin after coupling with NODAGA and radiolabeling with ⁶⁸Ga was in addition confirmed by autoradiography.

They have also been able to produce in particular a (F13A)Apelin-DOTA-⁶⁸Ga tracer with a radiochemical purity equal to or greater than 99%, and a (F13A)Apelin-NODA-Al¹⁸F tracer with a radiolabeling efficiency of 47%.

They also developed a Gallium-67 (⁶⁷Ga) radiolabeled tracer combining an Apelin, typically an Apelin corresponding to an amino acid sequence comprising or consisting in SEQ ID NO:1, SEQ ID NO: 2, SEQ ID NO: 3 or SEQ ID NO: 4, for example comprising anyone of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13, and a chelator (NODAGA) with which they performed in vitro saturation binding assay and in vitro internalization and efflux studies on colon carcinoma cells (T84 cell line).

Each of the herein described conjugated and labelled Apelin products advantageously binds a Apelin receptor protein also called “APJ” or “APLNR” or “angiotensin-like receptor”. APJ, first discovered as a new member of G protein-coupled receptors (GPCR) in 1993, is a 380 amino acid class A GPCR which shares 40-50% of the hydrophobic transmembrane regions with the angiotensin receptor (AT1) (Dowda B F O et al., 1993). Tatemoto et al. isolated in 1998 a APJ receptor ligand, which they named apelin. The human apelin receptor gene encodes for a protein (“APJ”) of 380 amino acids (SEQ ID NO: 18). Glu²⁰ and Asp²³ which are localized in the extracellular N-terminal tail of the APJ protein sequence, were first identified as crucial residues for binding of its endogenous ligand called apelin (Langelaan D N et al., 2013; Zhou N et al., 2003). In addition, Gerbier R et al. (2019) recently established that Asp⁹⁴, Glu¹⁷⁴ and Asp²⁸⁴ are also involved in apelin binding.

Thanks to the herein described labeled Apelin (typically conjugated and labeled Apelin) products, inventors herein demonstrate that the expression of the Apelin receptor protein (“APJ”) reflects angiogenesis, typically neoangiogenesis, and vasculogenesis within a subject and that the herein described labeled Apelin, which specifically recognizes and binds APJ can advantageously be used for labelling, detecting and/or imaging angiogenesis (typically neoangiogenesis), vasculogenesis or a tissue or organ expressing the APJ receptor, in particular a hypoxic tissue, for example a post-ischemic tissue or a tumor, in particular a cancerous tumor, the APJ receptor (“APJ”)-expressing tissue being typically a APJ-overexpressing tissue; for detecting, measuring, diagnosing, staging and/or monitoring angiogenesis (typically neoangiogenesis), vasculogenesis, an angiogenesis- and/or vasculogenesis-related disease or disorder, and/or a disease or disorder inducing or modulating the expression of a APJ receptor in a tissue or organ of a subject, in particular a disease or disorder associated to a tissue expressing APJ, in particular a hypoxic tissue, for example a post-ischemic tissue or a tumor, in particular a cancerous tumor, the APJ-expressing tissue being typically a APJ-overexpressing tissue; for preventing or treating angiogenesis, vasculogenesis, an angiogenesis- and/or vasculogenesis-related disease or disorder, and/or a disease or disorder inducing or modulating the expression of a APJ receptor in a tissue or organ in a subject, in particular a disease or disorder associated to a tissue expressing APJ, typically a hypoxic tissue, for example a post-ischemic tissue or a tumor, in particular a cancerous tumor, the APJ-expressing tissue being typically a APJ-overexpressing tissue; for determining the therapeutic eligibility of a human patient to a therapeutic treatment involving a particular agent or protocol; for evaluating or monitoring the therapeutic effect of an angiogenic or antiangiogenic treatment or of an APJ receptor-targeted treatment, or for determining the prognosis of a herein described disease or disorder, in a subject, preferably in a human patient. In other words, the herein described conjugated and labelled Apelin can advantageously be used as an APJ receptor-highly specific tracer or marker and/or as an angiogenesis/vasculogenesis-highly specific tracer or marker.

The herein described radiolabeled Apelin products, typically conjugated and labeled Apelin products, in addition advantageously exhibit favorable pharmacokinetics, particularly in terms of distribution, for example high tumor uptake, as well as rapid excretion through urinary and hepatic routes after intravenous (IV) injection.

In a particular aspect, the herein described labeled Apelin (typically conjugated and labeled Apelin) products can advantageously be used for evaluating tissue angiogenesis or vasculogenesis intensity, and/or as an early predictive factor of tissue perfusion, usable as soon as 1 day, typically 2 days, following the administration of an efficient treatment.

In the context of the present invention, the “subject” is an animal, typically a mammal. Examples of mammals include humans and non-human animals such as, without limitation, domesticated animals (e.g., cows, sheep, pigs, rabbits, cats, dogs, and horses), non-human primates (such as monkeys), and rodents (e.g., mice and rats). The “subject” is preferably a human being, whatever its gender, age, race or sex, and is typically a human patient.

Preferably, the radiolabeled Apelin is a radiolabeled human Apelin, typically a conjugated and radiolabeled human Apelin.

In a preferred embodiment, Apelin refers to a human protein, peptide or amino acid molecule containing about 12 to about 35 amino acids, preferably 13 amino acids. Apelin typically has an amino acid sequence comprising, or consisting in, a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

Apelin is typically encoded by a sequence selected from SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13. A preferred nucleic acid sequence is SEQ ID NO: 5 (“F13A”), SEQ ID NO: 7 (“Apelin-13”), SEQ ID NO: 8 (Pyr-Apelin-13”) or SEQ ID NO: 10 (“Apelin-13 (13[D-Phe]”).

Among previous amino acid sequences, SEQ ID NO: 5 is particularly preferred.

In a preferred aspect, the conjugated and labelled Apelin according to the invention is thus a conjugated and labelled human Apelin protein wherein Apelin consists in, or has an amino acid sequence consisting in, SEQ ID NO: 5 (F13A).

In other words, a preferred conjugated and labeled Apelin protein is selected from [⁶⁸Ga]Ga-NODAGA-SEQ ID NO: 5, [⁶⁸Ga]Ga-DOTA-SEQ ID NO: 5, [⁶⁸Ga]Ga-DOTAGA-SEQ ID NO: 5, [⁶⁸Ga]Ga-NOTA-SEQ ID NO: 5, [⁶⁸Ga]Ga-HBED-SEQ ID NO: 5, [⁶⁸Ga]Ga-DFO-SEQ ID NO: 5, [⁶⁸Ga]Ga-AAZTA-SEQ ID NO: 5, [⁶⁷Ga]Ga-NODAGA-SEQ ID NO: 5, [⁶⁷Ga]Ga-DOTA-SEQ ID NO: 5, [⁶⁷Ga]Ga-DOTAGA-SEQ ID NO: 5, [⁶⁷Ga]Ga-NOTA-SEQ ID NO: 5, [⁶⁷Ga]Ga-HBED-SEQ ID NO: 5, [⁶⁷Ga]Ga-DFO-SEQ ID NO: 5, [⁶⁷Ga]Ga-AAZTA-SEQ ID NO: 5 Al[¹⁸F]F-NOTA-SEQ ID NO: 5, Al[¹⁸F]F-NODA-SEQ ID NO: 5, Al[¹⁸F]F-DOTAGA-SEQ ID NO: 5, [⁶⁴Cu]Cu-DOTA-SEQ ID NO: 5, [⁶⁴Cu]Cu-DOTAGA-SEQ ID NO: 5, [⁸⁹Zr]Zr-DOTA-SEQ ID NO: 5, [⁸⁹Zr]Zr-DOTAGA-SEQ ID NO: 5, [¹⁷⁷Lu]Lu-DOTA-SEQ ID NO: 5, [¹⁷⁷Lu]Lu-DOTAGA-SEQ ID NO: 5, [¹⁷⁷Lu]Lu-DKFZ-SEQ ID NO: 5, [¹⁷⁷Lu]Lu-AAZTA-SEQ ID NO: 5, [²²⁵Ac]Ac-DOTA-SEQ ID NO: 5, [Pb²¹²]Pb-TCMC-SEQ ID NO: 5, [²¹³Bi]Bi-DTPA-SEQ ID NO: 5, [⁹⁰Y]Y-DTPA-SEQ ID NO: 5, [⁹⁰Y]Y-CHX-A″-DTPA-SEQ ID NO: 5, [¹¹¹In]In-DTPA-SEQ ID NO: 5, [¹⁴⁹Tb]Tb-DOTA-SEQ ID NO: 5, [¹⁴⁹Tb]Tb-DOTAGA-SEQ ID NO: 5, [¹⁵²Tb]Tb-DOTA-SEQ ID NO: 5, [¹⁵²Tb]Tb-DOTAGA-SEQ ID NO: 5, [⁵⁵Tb]Tb-DOTA-SEQ ID NO: 5, [¹⁵⁵Tb]Tb-DOTAGA-SEQ ID NO: 5, [¹⁶¹Tb]Tb-DOTA-SEQ ID NO: 5, [¹⁶¹Tb]Tb-DOTAGA-SEQ ID NO: 5, and any derivative thereof.

A particularly preferred conjugated and labeled Apelin protein is selected from [⁶⁸Ga]Ga-NODAGA-SEQ ID NO: 5, [⁶⁸Ga]Ga-DOTA-SEQ ID NO: 5, [⁶⁸Ga]Ga-DOTAGA-SEQ ID NO: 5, [⁶⁸Ga]Ga-HBED-SEQ ID NO: 5, [⁶⁸Ga]Ga-DFO-SEQ ID NO: 5, [¹⁸F]F-NOTA-SEQ ID NO: 5, [¹⁸F]F-NODA-SEQ ID NO: 5 and a derivative thereof. Such a particularly preferred conjugated and labeled Apelin protein is typically used as a tracer or is for use as a tracer.

Preferred F¹⁸-NOTA-SEQ ID NO: 5 derivatives are F¹⁸-maleimide-NOTA-SEQ ID NO: 5 and F¹⁸-propargyl-NOTA(tBu)₂-SEQ ID NO: 5.

Preferred F¹⁸-NODA-SEQ ID NO: 5 derivatives are F¹⁸—NCS-MP-NODA-SEQ ID NO: 5 and F¹⁸—NH₂-MPAA-NODA-SEQ ID NO: 5.

A particularly preferred conjugated and labeled Apelin proteins, in particular for use as a therapeutic agent, is selected from Y⁹⁰-DTPA-SEQ ID NO: 5, [¹⁷⁷Lu]Lu-DOTA-SEQ ID NO: 5, [¹⁷⁷Lu]Lu-DOTAGA-SEQ ID NO: 5, [¹⁷⁷Lu]Lu-DKFZ-SEQ ID NO: 5, [²²⁵Ac]Ac-DOTA-SEQ ID NO: 5, [Pb²¹²]Pb-TCMC-SEQ ID NO: 5, [²¹³Bi]Bi-DTPA-SEQ ID NO: 5, [⁹⁰Y]Y-DTPA-SEQ ID NO: 5, [⁹⁰Y]Y-CHX-A″-DTPA-SEQ ID NO: 5, [¹⁴⁹Tb]Tb-DOTA-SEQ ID NO: 5, [¹⁴⁹Tb]Tb-DOTAGA-SEQ ID NO: 5, [¹⁶¹Tb]Tb-DOTA-SEQ ID NO: 5, [¹⁶¹Tb]Tb-DOTAGA-SEQ ID NO: 5, and any derivative thereof, or from Y⁹⁰-DTPA-SEQ ID NO: 5, [¹⁷⁷Lu]Lu-DOTA-SEQ ID NO: 5, [¹⁷⁷Lu]Lu-DOTAGA-SEQ ID NO: 5, [¹⁷⁷Lu]Lu-DKFZ-SEQ ID NO: 5, [²²⁵Ac]Ac-DOTA-SEQ ID NO: 5, [Pb²¹²]Pb-TCMC-SEQ ID NO: 5, [²¹³Bi]Bi-DTPA-SEQ ID NO: 5, [⁹⁰Y]Y-DTPA-SEQ ID NO: 5, [⁹⁰Y]Y-CHX-A″-DTPA-SEQ ID NO: 5, [¹⁴⁹Tb]Tb-DOTA-SEQ ID NO: 5, [¹⁴⁹Tb]Tb-DOTAGA-SEQ ID NO: 5, [¹⁶¹Tb]Tb-DOTA-SEQ ID NO: 5, [¹⁶¹Tb]Tb-DOTAGA-SEQ ID NO: 5, and any derivative thereof.

In another aspect, the conjugated and labelled Apelin according to the invention is a conjugated and labelled human Apelin protein wherein Apelin comprises an amino acid sequence selected from SEQ ID NO: 1, 2, 3, or 4.

In other words, a preferred conjugated and labeled Apelin protein is selected from [⁶⁸Ga]Ga-NODAGA-SEQ ID NO: 1, 2, 3, or 4, [⁶⁸Ga]Ga-DOTA-SEQ ID NO: 1, 2, 3, or 4, [⁶⁸Ga]Ga-DOTAGA-SEQ ID NO: 1, 2, 3, or 4, [⁶⁸Ga]Ga-NOTA-SEQ ID NO: 1, 2, 3, or 4, [⁶⁸Ga]Ga-HBED-SEQ ID NO: 1, 2, 3, or 4, [⁶⁸Ga]Ga-DFO-SEQ ID NO: 1, 2, 3, or 4, [⁶⁸Ga]Ga-AAZTA-SEQ ID NO: 1, 2, 3, or 4, [⁶⁷Ga]Ga-NODAGA-SEQ ID NO: 1, 2, 3, or 4, [⁶⁷Ga]Ga-DOTA-SEQ ID NO: 1, 2, 3, or 4, [⁶⁷Ga]Ga-DOTAGA-SEQ ID NO: 1, 2, 3, or 4, [⁶⁷Ga]Ga-NOTA-SEQ ID NO: 1, 2, 3, or 4, [⁶⁷Ga]Ga-HBED-SEQ ID NO: 1, 2, 3, or 4, [⁶⁷Ga]Ga-DFO-SEQ ID NO: 1, 2, 3, or 4, [⁶⁷Ga]Ga-AAZTA-SEQ ID NO: 1, 2, 3, or 4, Al[¹⁸F]F-NOTA-SEQ ID NO: 1, 2, 3, or 4, Al[¹⁸F]F-NODA-SEQ ID NO: 1, 2, 3, or 4, Al[¹⁸F]F-DOTAGA-SEQ ID NO: 1, 2, 3, or 4, [⁶⁴Cu]Cu-DOTA-SEQ ID NO: 1, 2, 3, or 4, [⁶⁴Cu]Cu-DOTAGA-SEQ ID NO: 1, 2, 3, or 4, [⁸⁹Zr]Zr-DOTA-SEQ ID NO: 1, 2, 3, or 4, [⁸⁹Zr]Zr-DOTAGA-SEQ ID NO: 1, 2, 3, or 4, [¹⁷⁷Lu]Lu-DOTA-SEQ ID NO: 1, 2, 3, or 4, [¹⁷⁷Lu]Lu-DOTAGA-SEQ ID NO: 1, 2, 3, or 4, [¹⁷⁷Lu]Lu-DKFZ-SEQ ID NO: 1, 2, 3, or 4, [¹⁷⁷Lu]Lu-AAZTA-SEQ ID NO: 1, 2, 3, or 4, [²²⁵Ac]Ac-DOTA-SEQ ID NO: 1, 2, 3, or 4, [Pb²¹²]Pb-TCMC-SEQ ID NO: 1, 2, 3, or 4, [²¹³Bi]Bi-DTPA-SEQ ID NO: 1, 2, 3, or 4, [⁹⁰Y]Y-DTPA-SEQ ID NO: 1, 2, 3, or 4, [⁹⁰Y]Y-CHX-A″-DTPA-SEQ ID NO: 1, 2, 3, or 4, [¹¹¹In]In-DTPA-SEQ ID NO: 1, 2, 3, or 4, [¹⁴⁹Tb]Tb-DOTA-SEQ ID NO: 1, 2, 3, or 4, [¹⁴⁹Tb]Tb-DOTAGA-SEQ ID NO: 1, 2, 3, or 4, [¹⁵²Tb]Tb-DOTA-SEQ ID NO: 1, 2, 3, or 4, [¹⁵²Tb]Tb-DOTAGA-SEQ ID NO: 1, 2, 3, or 4, [¹⁵⁵Tb]Tb-DOTA-SEQ ID NO: 1, 2, 3, or 4, [¹⁵⁵Tb]Tb-DOTAGA-SEQ ID NO: 1, 2, 3, or 4, [¹⁶¹Tb]Tb-DOTA-SEQ ID NO: 1, 2, 3, or 4, [¹⁶¹Tb]Tb-DOTAGA-SEQ ID NO: 1, 2, 3, or 4, and any derivative thereof.

A particularly preferred conjugated and labeled Apelin protein is selected from [⁶⁸Ga]Ga-NODAGA-SEQ ID NO: 1, 2, 3 or 4, [⁶⁸Ga]Ga-DOTA-SEQ ID NO: 1, 2, 3 or 4, [⁶⁸Ga]Ga-DOTAGA-SEQ ID NO: 1, 2, 3 or 4, [⁶⁸Ga]Ga-HBED-SEQ ID NO: 1, 2, 3 or 4, [⁶⁸Ga]Ga-DFO-SEQ ID NO: 1, 2, 3 or 4, [¹⁸F]F-NOTA-SEQ ID NO: 1, 2, 3 or 4, [¹⁸F]F-NODA-SEQ ID NO: 1, 2, 3 or 4 and a derivative thereof. Such a particularly preferred conjugated and labeled Apelin protein is typically used as a tracer or is for use as a tracer.

Preferred F¹⁸-NOTA-SEQ ID NO: 1, 2, 3 or 4 derivatives are F¹⁸-maleimide-NOTA-SEQ ID NO: 1, 2, 3 or 4 and F¹⁸-propargyl-NOTA(tBu)₂-SEQ ID NO: 1, 2, 3 or 4.

Preferred F¹⁸-NODA-SEQ ID NO: 1, 2, 3 or 4 derivatives are F⁸—NCS-MP-NODA-SEQ ID NO: 1, 2, 3 or 4 and F¹⁸-NH₂-MPAA-NODA-SEQ ID NO: 1, 2, 3 or 4.

A particularly preferred conjugated and labeled Apelin proteins, in particular for use as a therapeutic agent, is selected from Y⁹⁰-DTPA-SEQ ID NO: 1, 2, 3, or 4, [¹⁷⁷Lu]Lu-DOTA-SEQ ID NO: 1, 2, 3, or 4, [¹⁷⁷Lu]Lu-DOTAGA-SEQ ID NO: 1, 2, 3, or 4, [¹⁷⁷Lu]Lu-DKFZ-SEQ ID NO: 1, 2, 3, or 4, [²²⁵Ac]Ac-DOTA-SEQ ID NO: 1, 2, 3, or 4, [Pb²¹²]Pb-TCMC-SEQ ID NO: 1, 2, 3, or 4, [²¹³Bi]Bi-DTPA-SEQ ID NO: 1, 2, 3, or 4, [⁹⁰Y]Y-DTPA-SEQ ID NO: 1, 2, 3, or 4, [⁹⁰Y]Y-CHX-A″-DTPA-SEQ ID NO: 1, 2, 3, or 4, [¹⁴⁹Tb]Tb-DOTA-SEQ ID NO: 1, 2, 3, or 4, [¹⁴⁹Tb]Tb-DOTAGA-SEQ ID NO: 1, 2, 3, or 4, [¹⁶¹Tb]Tb-DOTA-SEQ ID NO: 1, 2, 3, or 4, [¹⁶¹Tb]Tb-DOTAGA-SEQ ID NO: 1, 2, 3, or 4, and any derivative thereof.

A typical Apelin protein according to the present invention is, as explained previously, a protein capable of interacting specifically with a receptor of Apelin, typically APJ, in particular a sequence selected from SEQ ID NO: 4, 5, 12 and 13, preferably SEQ ID NO: 5.

In a particular aspect, the Apelin of interest selected in the context of the invention has been separated or recovered from a biological sample, typically from vessel, of a human subject, in particular from a vessel of a human subject suffering of ischemia, or from a tumor (cancerous tissue) sample of the subject. In a preferred aspect, said subject is a subject who is supposed to be exposed to a labeled Apelin according to the invention.

In another particular aspect, the Apelin of interest selected in the context of the invention is obtained using a method comprising the following steps of transfecting a mammalian cell with an appropriate vector expressing a Apelin protein, such as anyone of the herein described protein, and isolating the expressed Apelin protein.

The Apelin amino acid sequences of the present invention can be designed to be compatible with a diagnostic, therapeutic or prophylactic use, or with use in imaging, in a mammal, preferably in a human being. They can be, for example glycosylated, methylated, acetylated, phosphorylated, for targeting different types of tissues, in particular a pathological tissue such as, typically, an ischemic tissue or a solid tumor, preferably in a human being.

Suitable host cells for the expression of glycosylated human Apelin may be selected from mammalian cell lines, for example CHO cells.

In a particular aspect, the (conjugated and) labeled Apelin protein is thus glycosylated, methylated, acetylated, phosphorylated and/or fused to another polypeptide, such as a tag polypeptide sequence (for example a c-myc tag sequence).

In another preferred aspect, the (conjugated and) labeled Apelin according to the invention is compatible with an administration to a human subject, in particular by way of injection in the bloodstream.

Typically, the (conjugated and) labelled Apelin according to the invention is compatible with an intravenous, intracavitary or intraarterial administration to a human subject.

### Conjugates

In a preferred aspect of the present invention, any one of the herein described Apelin is labeled with a radioactive element and conjugated to a chelator or complexing agent or to a functional derivative thereof.

In the context of the invention, the chelator forming a conjugate compound with Apelin (the resulting product being also herein simply identified as “conjugate”) is typically selected from 6-amino-6 methylperhydro-1,4-diazepinetetraacetic acid (AAZTA), 1,4,7-triazacyclononane-1,4-diacetic acid (NODA), 1,4,7-triazacyclononane,1-glutaric acid-4,7 acetic acid (NODAGA), 1,4,7,10-tetraazacyclododecane-1,4,7,10-tetraacetic acid (DOTA), 2,2′,2″-(10-(2,6-dioxotetrahydro-2H-pyran-3-yl)-1,4,7,10-tetraazacyclododecane-1,4,7-triyl)triacetic acid (DOTAGA), 1,4,7-triazacyclononane-triacetic acid (NOTA), N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide (herein also identified as desferrioxamine or DFO), N,N′-Bis(2-hydroxybenzyl)-1-(4-bromoacetamidobenzyl)-1,2-ethylenediamine-N,N′-diacetic acid (HBED), triazacyclononane-phosphinate (TRAP), pentetic acid or diethylenetriaminepentaacetic acid (DTPA), bromoacetamidobenzyl(TETA),1,4,7-triazacyclononane-1,4-bis[methylene(hydroxymethyl)phosphinicacid]-7-[methylene(2-carboxyethyl)phosphinicacid])(NOPO), HBED-CC(DKFZ), 2-(4-isothiocyanotobenzyl)-1, 4, 7, 10-tetraaza-1, 4, 7, 10-tetra-(2-carbamonyl methyl)-cyclododecane (TCMC), N—[(R)-2-amino-3-(p-aminophenyl)propyl]-trans-(S,S)-cyclohexane-1,2-diamine-N,N,N′,N″,N″-pentaacetic acid (CHX-A″-DTPA) and a functional derivative thereof.

In a preferred aspect, the chelator is selected from NODAGA, DOTA, DOTAGA, AAZTA and NOTA.

In another preferred aspect, when the conjugated and labeled Apelin is for use as a tracer or contrast agent, the chelator is preferably selected from NODAGA, DOTA, DOTAGA, NOTA, HBED and DTPA. Particularly preferred chelators for use in imagery are NODAGA and DOTA.

In a further preferred aspect, when the conjugated and labeled Apelin is for use as a therapeutic agent, the chelator is preferably selected from DOTA, DKFZ, TCMC, DTPA and CHX-A″-DTPA. A particularly preferred chelator for use in therapy is DOTA.

1,4,7,10-tetraazacyclododecane-1,4,7,10-tetraacetic acid (also known as DOTA) is an organic compound with the formula (CH₂CH₂NCH₂CO₂H)₄. The molecule consists of a central 12-membered tetraaza (i.e., containing four nitrogen atoms) ring. DOTA is used as a complexing agent, especially for lanthanide ions. DOTA is derived from the macrocycle known as cyclen. The four secondary amine groups are modified by replacement of the N—H centers with N—CH₂CO₂H groups. The resulting aminopolycarboxylic acid, upon ionization of the carboxylic acid groups, is a high affinity chelating agent for di- and trivalent cations.

2,2′,2″-(10-(2,6-dioxotetrahydro-2H-pyran-3-yl)-1,4,7,10-tetraazacyclododecane-1,4,7-triyl)triacetic acid (also known as DOTAGA) is an organic compound with the formula C₂₁H₃₆N₅O₉. The molecule consists of a central 12-membered tetraaza (i.e., containing four nitrogen atoms) ring. DOTAGA is used as a complexing agent, especially for lanthanide ions. DOTAGA is derived from the functionalized macrocycle known as DOTA with an additional carboxyl group. The four secondary amine groups are modified by replacement of the N—H centers with N—CH₂CO₂H groups. The resulting aminopolycarboxylic acid, upon ionization of the carboxylic acid groups, is a high affinity chelating agent for di- and trivalent cations.

1,4,7-triazacyclononane,1-glutaric acid-4,7 acetic acid (also known as NODAGA) is an organic compound with the formula C₂₃H₃₁N₅O₇S. The molecule consists of a central 1,4,7-triazacyclononane (i.e. containing three nitrogen atoms) ring. NODAGA is used as a complexing agent, especially for lanthanide ions. NODAGA is derived from the macrocyle known as triazacyclononane. All secondary amine groups are modified by replacement of the N—H centers with N—CH₂CO₂H groups. The resulting aminopolycarboxylic acid, upon ionization of the carboxylic acid groups, is a high affinity chelating agent for di- and trivalent cations.

A functional derivative of a chelator or complexing agent as herein described designates any compound derived from the above-mentioned chelators or complexing agents by replacement of one or more of the functional groups thereof (i.e. groups involved in the chelating function) by another functional group without prejudice on said chelating function, and/or by addition and/or deletion or groups not involved in the chelating function without prejudice on said chelating function.

When present, the chelator may be linked directly to any one of the herein described Apelin or through a linker or spacer, the linker or spacer being easily selectable by the person skilled in the art. The linker or spacer is typically covalently coupled to both the chelator and Apelin.

Preferred NOTA derivatives include for example maleimide-NOTA [2,2′-(7-(2-((2-(2,5-dioxo-2,5-dihydro-1H-pyrrol-1-yl)ethyl)amino)-2-oxoethyl)-1,4,7-triazonane-1,4-diyl)diacetic acid] and propargyl-NOTA(tBu)₂ [di-tert-butyl 2,2′-(7-(2-oxo-2-(prop-2-yn-1-ylamino)ethyl)-1,4,7-triazonane-1,4-diyl)diacetate].

Preferred NODA derivatives include for example NCS-MP-NODA [2,2′-(7-(4-isothiocyanatobenzyl)-1,4,7-triazonane-1,4-diyl)diacetic acid] and NH₂-MPAA-NODA [2,2′-(7-(4-(2-((2-aminoethyl)amino)-2-oxoethyl)benzyl)-1,4,7-triazonane-1,4-diyl)diacetic acid].

Another example of chelator derivative of interest is p-NCS-Bz-DFO [N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide].

In a typical aspect of the invention, the radioactive element is a pharmaceutically acceptable radioactive element, i.e. a radionuclide adapted for use in medical imagery, preferably for use in PET and/or SPECT imagery, and/or for use in nuclear medicine therapy, typically radiotherapy.

The radioactive elements or radionuclides for use in medical imagery is typically a radioactive element having a short half-life (from about 1 min to 1, 2, 3 or 4 days) such as fluorine 18 (about 110 min), gallium 68 (about 67 min), indium 111 (67 h) and copper 64 (12.7 h). Photon- and low energy (inferior to 300 keV) gamma-emitting radionuclides are preferably used in the context of medical imagery.

The radioactive element or radionuclide typically used in nuclear medicine therapy is typically a radioactive element having an half-life between 1 day and 75 days such as lutetium 177 (6.64 h), actinium 225 (10 d), lead 212 (10.6 h), bismuth 213 (45 min), yttrium 90 (64.2 h) and Indium 111 (67 h). Bêta- or high energy gamma-emitting radionuclides are preferably used in the context of nuclear medicine therapy.

In a preferred aspect of the invention, the radioactive element is a radionuclide selected from gallium-68 (⁶⁸Ga), gallium-67 (⁶⁷Ga), lutetium-177 (¹⁷⁷Lu), fluorine-18 (F¹⁸), yttrium-90 (⁹⁰Y), bismuth-213 (²¹³Bi), actinium-225 (²²⁵Ac), lead-212 (²¹²Pb), indium-111 (¹¹¹In), zirconium-89 (⁸⁹Zr), terbium-149 (¹⁴⁹Tb), terbium-152 (¹⁵²Tb), terbium-155 (⁵⁵Tb), terbium-161 (¹⁶¹Tb) and copper-64 (⁶⁴Cu).

Gallium-68 (⁶⁸Ga), gallium-67 (⁶⁷Ga), fluorine-18 (F¹⁸), indium-111 (¹¹¹In) and copper-64 (⁶⁴Cu)) are preferably used in the context of imaging. A particularly preferred radionuclide is gallium-68 (⁶⁸Ga). Gallium-68 has a short half-life (67.71 min) and is a positron-emitting isotope. Another particularly preferred radionuclide is fluorine-18 (F¹⁸) which has a short half-life (109.7 minutes) and is a positron-emitting isotope. Another particularly preferred radionuclide is Gallium-67 which has a longer half-life (about 3.2 days) and is a single-photon emitting isotope.

Preferably, apelin is labeled with gallium-68 (⁶⁸Ga) or fluorine-18 (F¹⁸) when used in PET and is labeled with gallium-67 (⁶⁷Ga) when used in SPECT.

Lutetium-177 (¹⁷⁷Lu), actinium-225 (²²⁵Ac), lead-212 (²¹²Pb), bismuth-213 (²¹³Bi), yttrium-90 (⁹⁰Y) and indium-111 (¹¹¹In) are preferably used in the context of nuclear medicine therapy, typically radiotherapy.

The coupling between (any one of the herein described) Apelin and the selected radioactive element can be carried out using any chemical, biological or genetic technique known to those skilled in the art. The coupling typically involves one or more covalent, ionic, hydrogen, hydrophobic or Van der Waals bonds, preferably covalent and/or ionic bonds, and can occur at any site (including the N- and C-terminal sites) of the Apelin amino acid sequence having an adapted functional group such as —OH, —SH, —CO₂H, —NH₂, —SO₃H, —CN, —N₃, —NCS, —PO₂H, maleimide or succinimide ester, the functional group being naturally present or exogenously (i.e. artificially) introduced.

The radioactive element can be coupled directly to Apelin (synthesis in tandem) or indirectly via a linker or spacer. In a preferred embodiment, the radioactive element is linked to Apelin thanks to a chelating agent such as one of those herein above described.

In a particular aspect, conjugated and labeled Apelin are selected from [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin Al[¹⁸F]F-NOTA-Apelin, Al[¹⁸F]F-NODA-Apelin, Al[¹⁸F]F-DOTAGA-Apelin, [⁶⁴Cu]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [1′¹⁷⁷Lu]Lu-DOTA-Apelin, [¹⁷⁷Lu]Lu-DOTAGA-Apelin, [¹⁷⁷Lu]Lu-DKFZ-Apelin, [¹⁷⁷Lu]Lu-AAZTA-Apelin, [²²⁵Ac]Ac-DOTA-Apelin, [Pb²¹²]Pb-TCMC-Apelin, [²¹³Bi]Bi-DTPA-Apelin, [9′Y]Y-DTPA-Apelin, [⁹⁰′Y]Y-CHX-A″-DTPA-Apelin and [¹¹¹In]In-DTPA-Apelin, [¹⁴⁹Tb]Tb-DOTA-Apelin, [¹⁴⁹Tb]Tb-DOTAGA-Apelin, [¹⁵²Tb]Tb-DOTA-Apelin, [¹⁵²Tb]Tb-DOTAGA-Apelin, [⁵⁵Tb]Tb-DOTA-Apelin, [¹⁵⁵Tb]Tb-DOTAGA-Apelin, [¹⁶¹Tb]Tb-DOTA-Apelin, [¹⁶¹Tb]Tb-DOTAGA-Apelin, and any derivative thereof.

A particularly preferred radiolabeled F¹⁸-chelator-Apelin is selected from F¹⁸-maleimide-NOTA-Apelin, F¹⁸-propargyl-NOTA(tBu)₂-Apelin, F¹⁸—NCS-MP-NODA-Apelin, F¹⁸—NH₂-MPAA-NODA-Apelin and F¹⁸-p-NCS-Bz-DFO-Apelin.

Such labeled products are also known as radiotracers (also herein identified as “tracers”, “contrast agents” or “radiomarkers” or “radiotracers”) or as radiotherapeutic compounds depending on the intended use (imagery or therapy).

In a particular aspect, inventors indeed describe the use of a herein disclosed labeled Apelin, typically of a conjugated and labeled Apelin, as an imagery tracer, typically as a Single Photon Emission Computed Tomography (SPECT-CT) tracer or as a Positron Emission Tomography-Computed Tomography (PET-CT) tracer.

In a preferred aspect, inventors herein describe anyone of the herein above identified conjugated and labeled Apelin, in particular [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, [¹¹¹In]In-DTPA-Apelin, [Cu⁶⁴]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [⁵²Tb]Tb-DOTA-Apelin, [¹⁵²Tb]Tb-DOTAGA-Apelin, [⁵⁵Tb]Tb-DOTA-Apelin or [¹⁵⁵Tb]Tb-DOTAGA-Apelin for use in imagery, typically as an imagery tracer, preferably as a Single Photon Emission computed Tomography (SPECT-CT) tracer or as a Positron Emission Tomography-Computed Tomography (PET-CT) tracer. A preferred tracer is selected from [⁶⁸Ga]Ga-NODAGA-Apelin, [⁶⁸Ga]Ga-DFO-Apelin, Al[¹⁸F]F-NOTA-Apelin and Al[¹⁸F]F-NODA-Apelin.

A particularly preferred tracer is [⁶⁸Ga]Ga-NODAGA-Apelin.

Another particularly preferred tracer is [⁶⁸Ga]Ga-DOTA-Apelin.

Another particularly preferred tracer is Al[¹⁸F]F-maleimide-NOTA-Apelin, wherein maleimide-NOTA is 2,2′-(7-(2-((2-(2,5-dioxo-2,5-dihydro-1H-pyrrol-1-yl)ethyl)amino)-2-oxoethyl)-1,4,7-triazonane-1,4-diyl)diacetic acid.

Another particularly preferred tracer is Al[¹⁸F]F-propargyl-NOTA(tBu)₂-Apelin, wherein propargyl-NOTA(tBu)₂ is di-tert-butyl 2,2′-(7-(2-oxo-2-(prop-2-yn-1-ylamino)ethyl)-1,4,7-triazonane-1,4-diyl)diacetate.

A further particularly preferred tracer is Al[¹F]F-NCS-MP-NODA-Apelin, wherein NCS-MP-NODA is 2,2′-(7-(4-isothiocyanatobenzyl)-1,4,7-triazonane-1,4-diyl)diacetic acid.

A additional particularly preferred tracer is Al[¹F]F-NH₂-MPAA-NODA-Apelin, wherein NH₂-MPAA-NODA is 2,2′-(7-(4-(2-((2-aminoethyl)amino)-2-oxoethyl)benzyl)-1,4,7-triazonane-1,4-diyl)diacetic acid.

Another particularly preferred tracer is Al[¹⁸F]F-p-NCS-Bz-DFO-Apelin, wherein p-NCS-Bz-DFO is N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide.

In another particular aspect, inventors indeed describe the use of a herein disclosed labeled Apelin, typically of a conjugated and labeled Apelin, as a therapeutic agent, typically as a radiotherapeutic agent. Such a therapeutic agent is for use in nuclear medicine, typically in radiotherapy.

In a preferred aspect, inventors herein describe [¹⁷⁷Lu]Lu-DOTA-Apelin, [¹⁷⁷Lu]Lu-DOTAGA-Apelin, [¹⁷⁷Lu]Lu-DKFZ-Apelin, [²²⁵Ac]Ac-DOTA-Apelin, [Pb²¹²]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin, [¹⁴⁹Tb]Tb-DOTA-Apelin, [¹⁴⁹Tb]Tb-DOTAGA-Apelin, [¹⁶¹Tb]Tb-DOTA-Apelin, or [¹⁶¹Tb]Tb-DOTAGA-Apelin for use in therapy as a therapeutic agent, typically as a radiotherapeutic agent.

Preferred therapeutic agents are [¹⁷⁷Lu]Lu-DOTA-Apelin and [²²⁵Ac]Ac-DOTA-Apelin.

A particularly preferred therapeutic agent is [¹⁷⁷Lu]Lu-DOTA-Apelin.

The radiolabeled Apelin of the invention, typically the conjugated radiolabeled Apelin, can be in the form of any pharmaceutically acceptable (nontoxic) salts, hydrates, esters, solvates, precursors, metabolites or stereoisomers, these forms being well known by the skilled person of the art. The expression “pharmaceutically acceptable salts” designates any base or acid addition salts. Such a salt is generally prepared by reacting a free base with a suitable organic or inorganic acid. The salt may be a water-soluble or water-insoluble salt. These salts preserve the biological effectiveness and the properties of free bases. Examples thereof include typically ammonium acetate, hydrochloric acid and sodium acetate.

The invention also relates to a method of preparing a radiolabeled Apelin as claimed comprising a step of coupling Apelin to a radioactive element, preferably using a chelator or complexing agent such as one of those herein described. A particular method includes a step of coupling Apelin to a chelator or complexing agent before the step of coupling Apelin to a radioactive element.

A preferred final radiolabelled Apelin form is produced as sterile and apyrogenic solution diluted in saline.

In particular aspect, inventors herein describe the use of a radiolabeled Apelin as herein described, typically of a conjugated and radiolabeled Apelin as herein described, to prepare a composition for use for preventing or treating a disease, a disorder or a dysfunctional state as herein identified, typically angiogenesis, vasculogenesis, an angiogenesis- and/or vasculogenesis-associated disease, disorder or dysfunctional state, in particular a disease, disorder or dysfunctional state associated to a tissue expressing APJ, typically a hypoxic tissue, for example a post-ischemic tissue or a tumor, in particular a cancerous tumor, the APJ-expressing tissue being typically a APJ-overexpressing tissue.

In another particular aspect, inventors herein describe the use of a radiolabeled Apelin as herein described, typically of a conjugated and radiolabeled Apelin as herein described, to prepare a composition for use for preventing or treating a disease or disorder inducing the expression of a APJ receptor in a tissue or organ which does not express it in the healthy state, or a disease or disorder modulating (typically decreasing or increasing) the expression of a APJ receptor in a tissue or organ when compared to the expression of the APJ receptor observed in the healthy tissue or organ, for example a disease or disorder inducing the overexpression of a APJ receptor in a tissue or organ when compared to the expression of the APJ receptor observed in healthy a tissue or organ.

In particular aspect, inventors herein describe the use of radiolabeled Apelin according to the invention, typically of a conjugated and radiolabeled Apelin, to prepare a composition for use for preventing or treating angiogenesis, vasculogenesis, an angiogenesis- and/or vasculogenesis-related disease or disorder, and/or a disease or disorder inducing or modulating the expression of a APJ receptor in a tissue or organ; or for use for evaluating or monitoring the therapeutic effect of an angiogenic or antiangiogenic treatment, or of an APJ receptor-targeted treatment, in a subject.

### Compositions

Also herein disclosed are a composition, in particular a pharmaceutical composition comprising at least one radiolabeled Apelin, typically any conjugated and radiolabeled Apelin, as herein described, in a pharmaceutically acceptable diluent, excipient, carrier or support, and a method of preparing such a composition comprising providing a radiolabeled Apelin as herein described and formulating said compound with a suitable pharmaceutically acceptable diluent, excipient, carrier or support.

The expression “pharmaceutical composition” designates either i) a “diagnostic composition”, i.e. a composition for use (or used) in imaging (also herein identified as “imaging composition”) and/or in diagnostic, or ii) a “therapeutic composition”, i.e. a composition for use in prophylaxis (in the context of a preventive method applied to a subject in need thereof), or for use in therapy (in the context of a therapeutic method applied to a subject in need thereof, typically radiotherapy or molecular therapy, in particular radionuclide therapy), for example in cancer therapy.

In peptide receptor radionuclide therapy (PRRT), a cell-targeting protein (or peptide) is combined with a small amount of radioactive material, or radionuclide, creating a special type of radiopharmaceutical called a radiopeptide. When injected into the patient's bloodstream, this radiopeptide travels and binds to specific tumor cells, delivering a high dose of radiation to the cancer tissue while leaving the normal tissue unharmed.

The terms “treatment” or “therapy” refer to both therapeutic and prophylactic or preventive treatment or measures able to alleviate, slow progression (for example stop tumor growth) or cure a disease, disorder or dysfunctional state or related undesirable side effects.

Such a treatment or therapy is intended for a mammal subject, preferably a human being, in need thereof, as previously explained. Are considered as such, the subjects suffering from an angiogenesis- and/or vasculogenesis-related disease, disorder or dysfunctional state, in particular a disease, disorder or dysfunctional state associated to a tissue expressing APJ, typically over-expressing APJ, or those considered “at risk of developing” such a disease, disorder or dysfunctional state, in which this has to be prevented. Are also considered as such, the subjects suffering from a disease or disorder inducing or modulating the expression of a APJ receptor (“APJ”) in a tissue or organ, typically over-expressing APJ, or those considered “at risk of developing” such a disease, disorder or dysfunctional state, in which this has to be prevented. In a particular aspect, the angiogenesis- and/or vasculogenesis-related disease and/or the disease or disorder inducing or modulating the expression of a APJ receptor in a tissue or organ is a solid cancer or tumor, an ischemia-associated disease, disorder or dysfunctional state, atherosclerosis, an endothelial dysfunction-related disease, a cardiovascular disease or a metabolic disease as herein below described.

The conjugated and labelled Apelin is typically present in an effective amount in the composition. The expression “effective amount” respectively designates an amount or concentration sufficient to visualize and/or measure angiogenesis and/or vasculogenesis and/or to visualize API-expressing tissue(s) or organ(s) in the context of imaging, an amount or concentration sufficient to prevent angiogenesis and/or vasculogenesis in a preventive context, or an amount or concentration sufficient to attenuate or inhibit angiogenesis and/or vasculogenesis in a therapeutic context where angiogenesis and/or vasculogenesis is not desirable or even harmful such as cancer therapy.

A “labelling, marking or tracing effective amount” of a conjugated and labeled Apelin is an amount allowing the detection, imaging, measure, diagnosis or monitoring of a disease, disorder or dysfunctional state as herein below described, typically of a tissue or organ affected by such a disease, disorder or dysfunctional state, in a subject as herein defined, in particular in a mammal, preferably in a human being. Typical labelling, marking or tracing effective amounts for use in a mammal are between about 1 and 300 μg, for example between about 2 and 100 μg, preferably between about 20 and 50 μg. Typical amounts for use in a human being are between about 1 and 300 μg, preferably between about 10 and 200 μg, even more preferably between about 20 and 200 μg.

A “therapeutically effective amount” of a radiolabeled Apelin, typically of a conjugated and radiolabeled Apelin, according to the invention is an amount allowing the prevention or treatment of a disease, disorder or dysfunctional state, as herein described, in a subject as herein defined, in particular in a mammal, preferably in a human being. In such a context, the radiolabeled Apelin is typically used as a radiopeptide (as defined herein above).

Typical therapeutically effective amounts for use in a mammal, typically in a human being, are between about 5 and 1000 μg, preferably between about 25 and 500 μg, even more preferably between about 50 and 300 μg.

The dose of the labeled Apelin, typically of the conjugated and radiolabeled Apelin, in the diagnostic or pharmaceutical composition may be adjusted by the skilled person depending on the treated subject, the route of administration, the targeted tissue, the possible combination with an additional distinct biologically active compound or factor (as herein disclosed), etc.

A pharmaceutically acceptable excipient, vehicle or carrier, usable in a pharmaceutical composition of the invention is typically selected from saline and fillers such as sucrose, maltose, mannitol or trehalose. A pharmaceutically acceptable diluent, usable in the context of the present invention, typically in a diagnostic composition, is for example pharmaceutical grade saline.

The products of the invention (radiolabeled Apelin, typically conjugated and radiolabeled Apelin, and composition comprising such a radiolabeled Apelin) can be administered by any suitable route adapted to the intended use.

The product may be administered to a subject typically systemically, parenterally, or locally, for example subcutaneously, intraspinally, intraperitonally, intracerebrally or intratumoraly, given the targeted pathological tissue or area. Preferred modes of injection are systemic injection, in particular intra-venous or intra-arterial injection, and subcutaneous injection.

When for use in imaging and/or diagnostic, the product is typically administered to the subject by intra vascular route, preferably by intravenous or intratumoral injection, typically in the form of extemporaneous preparation/composition, preferably in the form of a sterile non pyrogenic solution for peripheral intravenous injection.

When for use in therapy, the product is typically administered to the subject in need thereof by intra vascular route, preferably by intravenous injection or by intratumoral injection, typically in the form of ready to use radiopharmaceutical composition, preferably in the form of sterile non pyrogenic solution for peripheral intravenous injection.

### Treatment

In a typical aspect, inventors herein describe a radiolabeled Apelin, typically a conjugated and radiolabeled Apelin, or a composition according to the invention comprising such a radiolabeled Apelin, for use for, or for use in, an in vitro, ex vivo or in vivo method of labelling, detecting and/or imaging angiogenesis, vasculogenesis or a tissue or organ expressing the APJ receptor; or for use for, or for use in, an in vitro, ex vivo or in vivo method detecting, measuring, diagnosing, staging and/or monitoring angiogenesis, vasculogenesis, an angiogenesis- and/or vasculogenesis-related disease or disorder, and/or a disease or disorder inducing or modulating the expression of a APJ receptor in a tissue or organ in a subject.

The products of the present invention (typically a radiolabeled Apelin as herein described or a composition comprising such a radiolabeled Apelin) may further be used in a method of diagnostic. The term diagnostic designates any in vivo, ex vivo or in vitro diagnosis, including typically APJ detection, imaging, monitoring, quantification, comparison, etc. In such a method, the radiolabeled Apelin is advantageously used as a biomarker providing an indication of the presence of a disease in a mammal, preferably a human being, in particular an ischemia or a cancer, of the presence of metastasis of a tumor, or of the evolution of such a diseased state. The measured value may be indeed compared to standard values associated to a healthy status of a subject. An overexpression of APJ may be, in particular, indicative of the presence of a solid cancer. A subexpression of APJ may be, in particular, indicative of the presence of angiogenesis.

In a further typical aspect, inventors herein describe a radiolabeled Apelin, typically any conjugated and radiolabeled Apelin as herein described, or a composition according to the invention comprising such a radiolabeled Apelin, for use for, or for use in a method of preventing angiogenesis, vasculogenesis, an angiogenesis- and/or vasculogenesis-related disease or disorder and/or a disease or disorder inducing or modulating the expression of a APJ receptor in a tissue or organ, in a subject, or for use for, or for use in, a method of treating angiogenesis, vasculogenesis, an angiogenesis- and/or vasculogenesis-related disease or disorder, and/or a disease or disorder inducing or modulating the expression of a APJ receptor in a tissue or organ, in a subject, in particular a disease or disorder associated to a tissue expressing APJ. The tissue expressing APJ is typically a hypoxic tissue, for example a post-ischemic tissue or a tumor tissue, in particular a cancerous tumor, the APJ-expressing tissue being typically a APJ-overexpressing tissue.

In another typical aspect, inventors herein provide a method of preventing or treating a disease, disorder or dysfunctional state in a mammal, preferably a human, as herein identified, in particular a method of preventing or treating angiogenesis, vasculogenesis, an angiogenesis- and/or vasculogenesis-related disease or disorder, and/or a disease or disorder inducing or modulating the expression of a APJ receptor in a tissue or organ, such as (a tissue) ischemia or a solid cancer, in particular a disease or disorder associated to a tissue expressing APJ, typically a hypoxic tissue, comprising administering to the mammal, a therapeutically effective amount of a radiolabeled Apelin or of a composition, as herein described, comprising such a radiolabeled Apelin protein.

In a typical method of preventing or treating a disease, the radiolabeled Apelin is intravascularly (for example intravenously) or intracavitaryly administered to the subject/patient (typically in the therapeutic sector of a nuclear medicine department). In a particular method, when exposed to the radiolabeled Apelin, the subject/patient is simultaneously administered with (for example infused with) a composition protecting the subject's kidneys, for example a solution of amino acids comprising typically lysine and arginine.

The radiolabeled Apelin of the invention advantageously allows a targeted radiotherapy which is typically directed against tissues expressing APJ, in particular against tissues over-expressing APJ. The radiolabeled Apelin of the invention is able to provide sufficient level of irradiation to targeted cells while not affecting surrounding tissues, typically healthy tissues. Thus, radiation therapy using the radiolabeled Apelin of the invention will allow more specific, effective as well as shorter treatments and will advantageously induce fewer detrimental side effects for the treated patient.

In a further typical aspect, inventors herein describe a radiolabeled Apelin, typically any conjugated and radiolabeled Apelin as herein described, or a composition according to the invention comprising such a radiolabeled Apelin, for use for, or for use in, a method of evaluating or monitoring the therapeutic effect of an angiogenic or anti-angiogenic treatment, or of an APJ receptor-targeted treatment, in a subject.

In a further typical aspect, inventors herein provide a method of evaluating or monitoring the therapeutic effect of an angiogenic or antiangiogenic treatment or of an APJ receptor-targeted treatment in a subject.

Also herein disclosed are a method for labelling, detecting and/or imaging angiogenesis, vasculogenesis or a tissue or organ expressing, typically overexpressing, the APJ receptor, and a method for detecting, measuring, diagnosing, staging and/or monitoring angiogenesis, vasculogenesis, an angiogenesis- and/or vasculogenesis-related disease or disorder, and/or a disease or disorder inducing or modulating the expression of a APJ receptor in a tissue or organ in a subject, comprising using a radiolabeled Apelin as herein described. The method typically comprises i) administering to the subject a radiolabeled Apelin as herein described, ii) performing an imaging method, and iii) determining or analysing the presence and/or amount of said radiolabeled Apelin. The presence of a signal is typically indicative of the presence of angiogenesis, or of a APJ overexpressing tissue, typically of a cancerous tissue, and/or indicates the level of angiogenesis or the stage of disease or disorder as herein described such as cancer. The term “analysing” refers to any method which allows determining if a signal corresponds to a normal signal or not. The analyses may not only be visual but also involve quantitative analyses. The analyses may include steps of comparing the value of a signal obtained by imagery to the value of the signal of a known healthy tissue of the same subject or of another (reference) subject or population. The value of the signal may also be compared to a reference value. A value more important than a reference value or a control from a healthy tissue is indicative of the presence of angiogenesis/vasculogenesis and possibly of cancerous cells. The stage of evolution of a cancer may be assessed by comparing in a same subject a signal obtained after two different imaging spaced in time.

In an imaging and/or diagnostic context, the radiolabeled Apelin is administrated before performing a PET-CT or SPECT-CT Scan on the subject (typically in the diagnostic sector of a nuclear medicine department).

The radiolabeled Apelin of the invention advantageously allows an effective, reliable and selective labeling of tissues expressing APJ, in particular of APJ over-expressing tissues.

The invention shows that the radiolabeled Apelin of the invention retains the ability to effectively bind APJ-expressing cells, in particular APJ-overexpressing cells.

The radiolabeled Apelin of the invention is in addition capable of discriminating APJ-overexpressing cells from cells expressing regular levels of APJ (e.g. normal tissue). In other words, the radioactive element portion of the claimed radiolabeled Apelin will preferentially mark APJ-overexpressing cells.

Though similar to angiogenesis, vasculogenesis is different in one aspect: the terms angiogenesis (and neoangiogenesis) denotes the formation of new blood vessels from pre-existing ones, whereas vasculogenesis is the term used for the formation of new blood vessels when there are no pre-existing ones. For example, if a monolayer of endothelial cells begins sprouting to form capillaries, angiogenesis is occurring. Vasculogenesis, in contrast, is when endothelial precursor cells (angioblasts) migrate and differentiate in response to local cues (such as growth factors and extracellular matrices) to form new blood vessels. These vascular trees are then pruned and extended through angiogenesis.

An angiogenesis- or vasculogenesis-related disease, disorder or dysfunctional state is a disease, disorder or dysfunctional state leading to abnormal vasculogenesis and/or angiogenesis, in particular a disease, disorder or dysfunctional state leading to tissue ischemia, to an undesirable neovascularization, to vascular permeability (alteration of the intercellular junctions of endothelial cells) and/or vascular endothelial cell growth. Examples of such disease include cancer, typically solid cancer or solid cancerous tumor; diabetes; age-related macular degeneration (also herein identified as “macular degeneration”); rheumatoid arthritis; psoriasis; any known vascular diseases including atherosclerotic vascular disease (also herein identified as “atherosclerosis”), cardiovascular disease such as coronary artery disease, ischemic heart disease, in particular myocardial ischemia or stroke, cerebrovascular ischemia, peripheral vascular disease such as peripheral artery occlusive disease.

In these conditions leading to an undesirable neovascularization, new blood vessels feed diseased tissues, destroy normal tissues, and in the case of cancer, the new vessels allow the growth of the cancerous tumor and/or allow tumor cells to escape into the circulation and lodge in other organs (tumor metastases).

Disorders may be the consequence of a disease as described above or of a trauma. Typical disorders are for example inflammation, edema, fibrosis and necrosis.

Examples of relevant dysfunctional states, diseases or disorders are, or affect a tissue or organ, characterized by a lack of or, on the contrary, an excessive expression of a receptor for Apelin, in particular APJ, compared to standard expression thereof (the standard expression being that of a reference population or subject, typically of a healthy population or subject/tissue or organ). Dysfunctional states characterized by an excessive expression of APJ, such as a solid cancer, are advantageously treated by a radiolabeled Apelin as herein described, typically a conjugated and radiolabeled Apelin, or a therapeutic composition comprising such a labeled Apelin, typically in the context of nuclear medicine therapy according to a method as herein above described.

In a particular aspect, the angiogenesis- and/or vasculogenesis-related disease or disorder, or the disease or disorder inducing or modulating the expression of a APJ receptor in a tissue or organ, is selected from, ischemia or an ischemia-associated disease or disorder, a solid cancer or solid cancerous tumor, atherosclerosis, an endothelial dysfunction-related disease, a cardiovascular disease, and a metabolic disease such as diabetes mellitus and obesity, preferably from ischemia or an ischemia-associated disease or disorder, a solid cancer or solid cancerous tumor, atherosclerosis, a cardiovascular disease, and a metabolic disease.

The solid cancer is typically a cancer wherein the cancerous tumor and/or cancerous tumor vasculature expresses the APJ receptor (“APJ”). The solid cancer is typically selected from lung cancer, cholangiocarcinoma, liver cancer, gastric cancer, prostate cancer, ovarian cancer, breast cancer, renal cancer, squamous cell carcinoma, multiple myeloma, glioblastoma, colon cancer in particular obesity-related colon cancer, and endometrial cancer in particular obesity-related endometrial cancer.

A typical example of endothelial dysfunction-related disease is an endothelial dysfunction associated to fibrosis or to a cardiovascular disease.

Non-restrictive typical examples of cardiovascular diseases are atherosclerosis, hypertension, heart failure, myocardial infarction, stroke, a retinopathy and an arteriopathy.

When the herein described therapeutic composition is for use for, or for use in a method of, preventing and/or treating cancer, the composition may further comprise, in addition to the at least one herein described radiolabeled Apelin, typically conjugated and radiolabeled Apelin, at least one anti-angiogenic agent (i.e. a biologically active factor which inhibits or interferes with blood vessel development), at least one distinct anti-cancer agent or drug, such as a chemotherapeutic drug which will be easily selected by the skilled person depending typically on the cancer to be treated or cancer metastases to be prevented, and/or at least one APJ receptor-targeted treatment.

Anti-angiogenic factors usable in the context of the present invention may be selected from an antibody directed against an angiogenic factor as previously defined, angioarrestin, angiostatin (plasminogen fragment), antiangiogenic antithrombin III, cartilage-derived inhibitor (CDI), CD59 complement fragment, endostatin (collagen XVIII fragment), fibronectin fragment, gro-beta, an heparinase, heparin hexasaccharide fragment, human chorionic gonadotropin (hCG), interferon alpha/beta/gamma, Interferon inducible protein (IP-10), interleukin-12, kringle 5 (plasminogen fragment), metalloproteinase inhibitors (TIMPs), 2-Methoxyestradiol, placental ribonuclease inhibitor, plasminogen activator inhibitor, platelet factor-4 (PF4), prolactin 16 kD fragment, proliferin-related protein (PRP), a retinoid, tetrahydrocortisol-S, thrombospondin-1 (TSP-1), transforming growth factor-beta (TGF-b), vasculostatin, vasostatin (calreticulin fragment), etc., and a mixture thereof.

APJ receptor-targeted factors usable in the context of the present invention may be selected from a therapeutic agent as herein described by inventors for the first time, in particular [¹⁷⁷Lu]Lu-DOTA-Apelin, [¹⁷⁷Lu]Lu-DOTAGA-Apelin, [¹⁷⁷Lu]Lu-DKFZ-Apelin, [¹⁷⁷Lu]Lu-AAZTA-Apelin [²²⁵Ac]Ac-DOTA-Apelin, [Pb²¹²]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin, [¹⁴⁹Tb]Tb-DOTA-Apelin, [¹⁴⁹Tb]Tb-DOTAGA-Apelin, [¹⁶¹Tb]Tb-DOTA-Apelin, or [¹⁶¹Tb]Tb-DOTAGA-Apelin; an antibody, typically a monoclonal antibody directed against APJ, an antagonist for use in oncology as described in Picault et al. (2014) or in Hall C et al. (2017) such as F13A or ML221, and an agonist for use for treating a vascular related disease as described in Xin Q et al. (2015) or in Schreiber C A et al. (2016) such as Apelin-13 or ELABELA.

In the context of the invention, ischemia is typically a peripheral ischemia (such as a peripheral artery occlusive disease), a myocardial ischemia or a cerebral ischemia (also herein identified as cerebrovascular ischemia), such as stroke.

The ischemia-associated disease or disorder is preferably peripheral and/or myocardial ischemia.

When the herein described therapeutic composition is for use for, or for use in a method of, preventing and/or treating ischemia or an ischemia-associated disease or disorder, the composition may further comprise, in addition to at least one herein described labeled Apelin, typically conjugated and labeled Apelin, at least one distinct angiogenic factor (i.e. a factor which favors blood vessel development).

Angiogenic factors usable in the context of the present invention may be selected from angiogenin, angiopoietin-1, Del-1, fibroblast growth factors: acidic (aFGF) and basic (bFGF), follistatin, granulocyte colony-stimulating factor (G-CSF), granulocyte-macrophage colony stimulating factor (GM-CSF), stem cell factor (SCF), hepatocyte growth factor (HGF)/scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), erytropoietin (EPO), endothelial nitric oxyd synthase (e-NOS), progranulin, proliferin, transforming growth factor-alpha (TGF-alpha), transforming growth factor-beta (TGF-beta), tumor necrosis factor-alpha (TNF-alpha), vascular endothelial growth factor (VEGF), vascular permeability factor (VPF), Angiopoietin-1 (Ang1), plasminogen activator urokinase (PLAU/u-Pa), the matrix metallopeptidase MMP-2, the VEGF receptor 2 (KDR), stromal-cell-derived-factor-1 (SDF-1), etc., and a mixture thereof.

Preferred angiogenic factors may be selected from vascular endothelial growth factor (VEGF—see experimental section and FIG. 5), stromal-cell-derived-factor-1 (SDF-1), basic fibroblast growth factors (bFGF), erythropoietin (EPO), granulocyte colony-stimulating factor (G-CSF), granulocyte-macrophage colony stimulating factor (GM-CSF), stem cell factor (SCF), interleukin-8 (IL-8) and a mixture thereof.

Various protocols may be used for the administration to the subject, such as simultaneous or sequential administration of the radiolabeled Apelin, typically of the conjugated and radiolabeled Apelin, and of any other compound as identified previously, single or repeated administration, etc., which may be adjusted by the skilled person.

An additional object herein described is a kit comprising Apelin, in particular an Apelin comprising SEQ ID NO: 1, SEQ ID NO: 2 or SEQ ID NO:3 (for example SEQ ID NO: 4, 5 or 6), a radioactive element and preferably a chelator, in two, three or more distinct containers, or a Apelin-chelator conjugate in a single container and a radioactive element in a distinct container, and optionally a leaflet providing guidelines.

The kit may further comprise a reducing agent such as for example stannous chloride, a buffer for pH adjustment such as for example sodium acetate or ammonium acetate, and/or a sterile non pyrogenic solution.

Another particular kit may further comprise a solution of amino acids comprising typically at least lysine and/or arginine.

Further herein described is the use of a herein described kit for producing a labeled Apelin as herein described, typically a conjugated and labeled Apelin, or for implementing anyone of the herein described uses or methods.

The examples, which follow, and their corresponding figures illustrate the invention without limiting the scope thereof.

### FIGURES

FIG. 1. Radiochemical purity in reactional medium and in human serum for two hours.

FIG. 2. APJ expression appreciated by Western-Blot corrected by GAPDH expression in different cell lines.

FIG. 3. T84 autoradiography in baseline or blocking conditions.

FIG. 4. Organs biodistribution of [⁶⁸Ga]Ga-NODAGA-Apelin13(F13A) in murine model (n=3).

FIG. 5. PET images of [⁶⁸Ga]Ga-NODAGA-Apelin13(F13A) biodistribution in healthy mice for two hours.

FIG. 6. Tracers [⁶⁸Ga]Ga-NODAGA-Apelin13(F13A) and [⁶⁸Ga]Ga-NODAGA-RGD₂ accumulation in Matrigel.

FIG. 7. [⁶⁸Ga]Ga-NODAGA-Apelin13(F13A) PET images in Matrigel over 21 Days.

FIG. 8. Ischemic limb doppler signal and non-ischemic limb doppler signal ratio over time.

FIG. 9. [⁶⁸Ga]Ga-NODAGA-Apelin13(F13A) and [⁶⁸Ga]Ga-NODAGA-RGD₂ targeting in ischemic limb (corrected by non-ischemic limb signal) over time.

FIG. 10. Representative [⁶⁸Ga]Ga-NODAGA-Apelin13(F13A) PET images over 21 days in mouse model of hindlimb ischemia.

FIG. 11. Correlation of [⁶⁸Ga]Ga-NODAGA-Apelin13(F13A) PET signal ratio on day 7 and LASER-Doppler signal at the day of surgery (left); Correlation of [⁶⁸Ga]Ga-NODAGA-Apelin13(F13A) PET signal ratio on day 7 and reperfusion on day 21 (right).

FIG. 12. [⁶⁸Ga]Ga-NODAGA-Apelin13(F13A) PET signal on day 7 and day-input function (LASER Doppler Day7/Day0) uncorrelation.

FIG. 13. Tumor to muscle [⁶⁸Ga]Ga-NODAGA-Apelin13(F13A) PET signal ratio in baseline or in blocking conditions in ectopic mice model of human colon adenocarcinoma.

FIG. 14. [⁶⁸Ga]Ga-NODAGA-Apelin13(F13A) PET images in baseline or in blocking conditions in ectopic mice model of human colon adenocarcinoma.

FIG. 15. Tumor to background [68Ga]Ga-NODAGA-Apelin13(F13A) PET signal ratio and [⁶⁸Ga]Ga-NODAGA-RGD₂ PET signal ratio in ectopic mice model of human colon adenocarcinoma.

FIG. 16. Representative radioHPLC chromatogram of [⁶⁸Ga]Ga-AP747.

FIG. 17. Radio-TLC chromatogram of [⁶⁷Ga]Ga-AP747.

FIG. 18. Saturation binding curve of [⁶⁷Ga]Ga-AP747 towards APJ receptor on T84 cells.

FIG. 19. APJ receptor specific internalized and membrane bound fraction of [⁶⁷Ga]Ga-AP747 in T84 cells.

FIG. 20. Cellular efflux of [⁶⁷Ga]Ga-AP747 on T84 cells.

FIG. 21. Radiochromatogram of [⁶⁸Ga]Ga-DOTA-Apelin-F13A (Rf0) showing excellent radiochemical purity ≥99% after purification with neglectable [⁶⁸Ga]Ga³⁺ at the front (Rf1). RadioTLC: ITLC/sg paper in 0.1M sodium citrate solution pH=5.

FIG. 22. (A): MicroPET signal quantification of [⁶⁸Ga]Ga-AP747 and [⁶⁸Ga]Ga-RGD₂ expressed as an ipsi—to contralateral signal ratio. (B): Representative images of [⁶⁸Ga]Ga-AP747 and [⁶⁸Ga]Ga-RGD₂ microPET/CT in the same animal. Dotted orange lines represent both hemispheres.

FIG. 23. (A) Representative image of [⁶⁸Ga]Ga-AP747 microPET/CT 3 days after MCAO in rat. (B) Quantification of [⁶⁸Ga]Ga-AP747 microPET/CT signal quantification in ipsi- and contralateral hemisphere before MCAO, on the day of MCAO, and every day for 7 days after MCAO in rat.

### EXAMPLES

### Example 1

### Materials and Methods

### NODAGA Conjugation

Apelin-13(F13A), purchased from Sigma-Aldrich (Merck Millipore) was solubilized in 0.2M bicarbonate buffer (1 mg/ml) and added to 10 equivalents of NODA-GA-NHS ester (CheMatech) in 0.2M bicarbonate buffer. The mixture was left at room temperature (RT) for 2 h. The conjugate was then transferred to a tC18 Cartridge (Sep-Pak) washed 2 times with water to eliminate unreacted small molecules then eluted with EtOH. Solvent was evaporated at RT, PBS was added and conjugate in PBS stored at −20° C.

### Radiochemistry

Gallium was obtained in ⁶⁸GaCl₃ form using a commercial TiO₂-based ⁶⁸Ge/⁶⁸Ga generator (Galliapharm, Eckert&Ziegler). ⁶⁸GaCl₃ (200.69±40.97 MBq/0.5 mL) was eluted from a ⁶⁸Ge/⁶⁸Ga generator using 0.1 N HCl, after which 4M ammonium acetate buffer (pH 7.4) was added. This solution was then added to NODAGA-Apelin-13(F13A) (1 μg/μL); final pH of the mixture was 6.0. The reaction mixture was incubated at RT for 5 min.

Determination of radiochemical purity was done by radio-thin-layer chromatography (ITLC-SG) and was performed using a Ray-test miniGITA radio-TLC scanner detector (Straubenhardt, Ge) (eluents, 1:1 [v/v] mixture of 1M aqueous ammonium acetate solution and methanol and also in Trisodium Citrate 0,1M). Evaluation of ⁶⁸Ga-NODAGA-Apelin13(F13A) stability was performed in human serum at 60 and 120 min after radiosynthesis.

NODAGA-RGD was purchased from ABX and radiolabelled as recommended with ⁶⁸Ga by the manufacturer.

### In Vitro Experiments

### Western Blot

APJ expression was evaluated by Western-Blot with cell lysates. Cell lysates were loaded on polyacrylamide gel (NuPAGE, Invitrogen, 4%-12%). After migration (80V, 30 minutes), proteins were transferred to nitrocellulose membrane (checked by Rouge-Ponceau). Membrane was saturated (TBST-3%; BSA, Tris-buffered saline Tween 20%; Bovine Serum Albumine 3%) and then Anti-APJ Apelin Receptor Antibody: 5H5L9 (rabbit monoclonal Invitrogen, 1 μg/mL) was added overnight, under agitation. After TBST wash, secondary antibody: Goat Anti-Rabbit HRP-tagged (Thermofisher) was added for one hour. Chemiluminescent revelation was made thanks to ECL kit (Thermofischer). Membrane images acquisition were performed by Gbox (Syngene). Finally, a stripping was performed to determine GADPH expression.

### Autoradiography

A blocking strategy was performed on cells expressing the highest level of APJ. This strategy consists in adding a large excess (100-fold) of unconjugated peptide (Apelin13(F13A)) before adding inventors' product of interest. Unspecific tracer was eliminated by several washes. The remaining activity, considered as specifically bound to the target, was evaluated by autoradiography.

### Human Umbilical Vein Endothelial Cells

HUVEC cell lines (Laboratoire de Thérapie cellulaire, CHU La Conception AP-HM/C2VN Aix-Marseille Université) were cultivated in EGM-2 medium complemented with 10% fetal bovine serum decomplemented and 1% antimycotic-antibiotic mix. Cell lines were maintained in a humidified 5% CO₂ incubator at 37° C. HUVEC's activation was performed by incubation with TNF-alpha (10 ng/ml) overnight.

### Human Colon Adenocarcinoma Cell Line

T84 cell line (EuroBioDev) was cultivated in DMEM-F12/Glutamax medium complemented with 10% fetal bovine serum decomplemented and 1% antimycotic-antibiotic mix. Cell lines were maintained in a humidified 5% CO₂ incubator at 37° C.

### Human Glioblastoma Cell Line

U87 cell line was cultivated in Dulbecco's modified Eagle's medium complemented with 10% fetal bovine serum, 1% antimycotic-antibiotic mix, and 1% non-essential amino-acid. Cell lines were maintained in a humidified 5% CO₂ incubator at 37° C.

### Human Pancreatic Adenocarcinoma Cell Line

SOJ6 cell line (CRCM, Aix-Marseille Université) was cultivated in DMEM-F12/Glutamax/Pyruvate complemented with 10% fetal bovine serum decomplemented and 1% antimycotic-antibiotic mix. Cell lines were maintained in a humidified 5% CO₂ incubator at 37° C.

### Animal Experiments

All procedures using animals were approved by the Institution's Animal Care and Use Committee (CE71, Aix-Marseille Université) and were conducted according to the 2010/63/EU European Union Directive. Swiss and Swiss Nude mice were housed in enriched cages placed in a temperature- and hygrometry-controlled room with daily monitoring and fed with water and commercial diet ad libitum.

### Mouse Model of Hindlimb Ischemia and Matrigel

Unilateral hindlimb ischemia was performed on 9-week-old male Swiss mice (Janvier Labs) after femoral artery excision under 2% isoflurane anesthesia. LASER Doppler perfusion imaging (Perimed, Craponne, France) was used to assess revascularization from day 0 to day 21 after surgery. Perfusion results are expressed as a ratio of ischemic to non-ischemic limb blood flow. Hindlimb ischemic damage was quantified on Days 1, 3, 7, 10, 13 and 21.

These mice were also subcutaneously implanted with Matrigel (Dutscher) supplemented with 10% fetal bovine serum under 2% isoflurane anesthesia.

### Ectopic Mouse Model of Human Colon Adenocarcinoma

Human colon adenocarcinoma xenografts were established by subcutaneous injections of 1×10⁶ T84 cells into 6-week-old male Swiss nude mice (Charles River) under 2% isoflurane anesthesia.

### MicroPET Imaging

On hindlimb ischemia mouse model (n=8) and Matrigel mouse model (n=7) on day 1, 3, 7, 10, 13 and 21 post-surgery, mice were IV injected with 5-10 MBq of ⁶⁸Ga-NODAGA-Apelin13(F13A) under 2% isoflurane anesthesia.

PET images were acquired 60 min after IV injection on a Mediso Nanoscan PET/CT under 2% isoflurane anesthesia. On hindlimb ischemia mouse model and on day 1, 3, 7, 10, 13 and 21 post-surgery, mice were IV injected with 5-10 MBq of [⁶⁸Ga]Ga-NODAGA-RGD₂ injection under 2% isoflurane anesthesia. PET images were acquired 60 min after IV injection on a Mediso Nanoscan PET/CT under 2% isoflurane anesthesia.

For blocking experiments, a 50-fold excess of unconjugated peptide: Apelin-13(F13A) was IV injected 30 min previous ⁶⁸Ga-NODAGA-Apelin13(F13A), and PET images were acquired 1 hour 30 min after the first IV injection on a Mediso Nanoscan PET/CT under 2% isoflurane anesthesia.

For biodistribution study in healthy mice (n=3), images were continuously acquired just after ⁶⁸Ga-NODAGA-Apelin13(F13A) IV injection with 5-6 MBq and recorded up to 2 h post injection on a Mediso NanoPET/CT under 2% isoflurane anesthesia.

On colon adenocarcinoma mice model (n=3), mice were IV injected with 5-10 MBq of [⁶⁸Ga]Ga-NODAGA-RGD₂ or ⁶⁸Ga-NODAGA-Apelin13(F13A) respectively under anesthesia.

PET images were acquired 1 h after IV injection on a Mediso NanoPET/CT under 2% isoflurane anesthesia.

Quantitative region-of-interest (ROI) analysis of the PET images was performed on attenuation- and decay-corrected PET images using InVicro—VivoQuant software and tissue uptake values are presented as an ischemic muscle to contralateral muscle ratio and as a percentage of the injected dose per gram of tissue (% ID/g) which was determined by decay correction for each sample normalized to a standard of known weight, which was representative of the injected dose.

### Statistical Analysis

Biodistribution data were analysed using the Graphpad Prism software (San Diego, Calif.). Data are presented as mean values±SD. Ischemic to contralateral muscle ratio were analyzed using the two-way analysis of variance (ANOVA) and no parametric t-test (Mann Whitney test). Differences were considered statistically significant when p<0.05.

### Results

### Radiochemistry

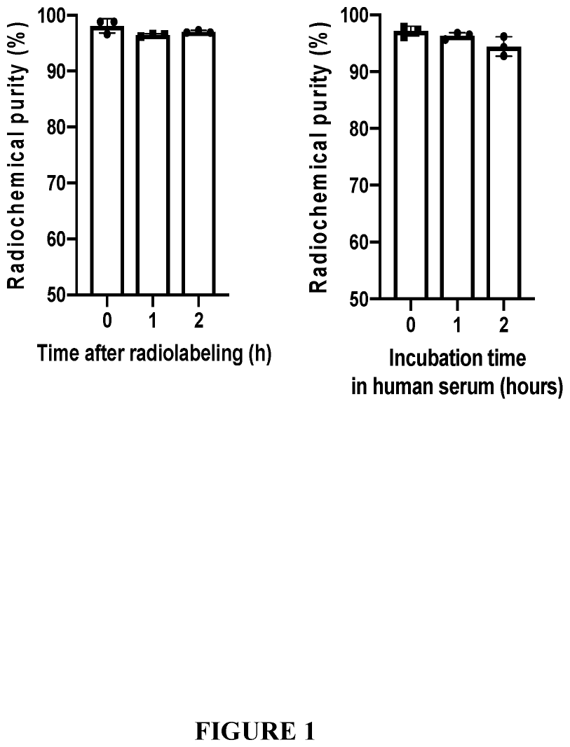

Incubation in human serum didn't lead to significant tampering of [⁶⁸Ga]-NODAGA-Apelin13(F13A) radiochemical purity until two hours post-incubation (n=3). Radiolabeling remained stable over time in molecular imaging conditions (<2 h) (FIG. 1).

### In Vitro Experiments

### Western Blot

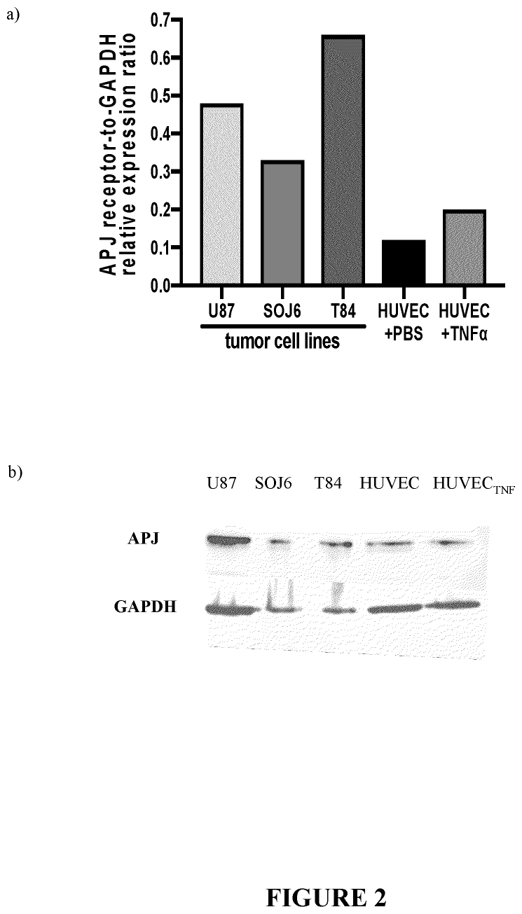

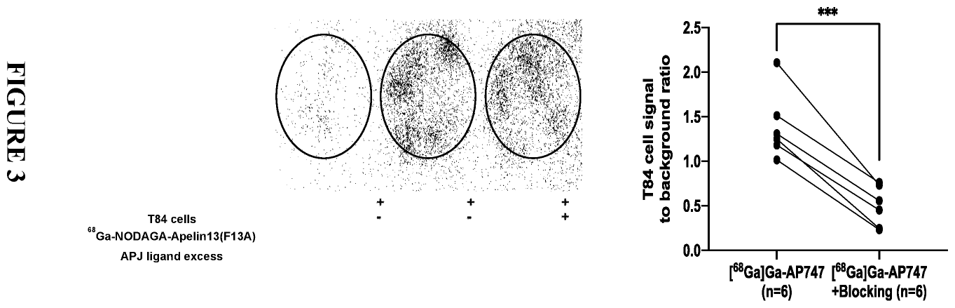

In order to evaluate tissue APJ expression in different cell lines Western Blot was performed (FIG. 2) on U87, SOJ6, T84, HUVECs and TNFα-activated HUVECs. T84 cell line seems to be the line with the higher expression of APJ. Moreover, activated HUVECs express a higher level of APJ than HUVECs in baseline conditions. APJ expression evaluation was corrected by the expression of GADPH (charge indicator).

### Autoradiography

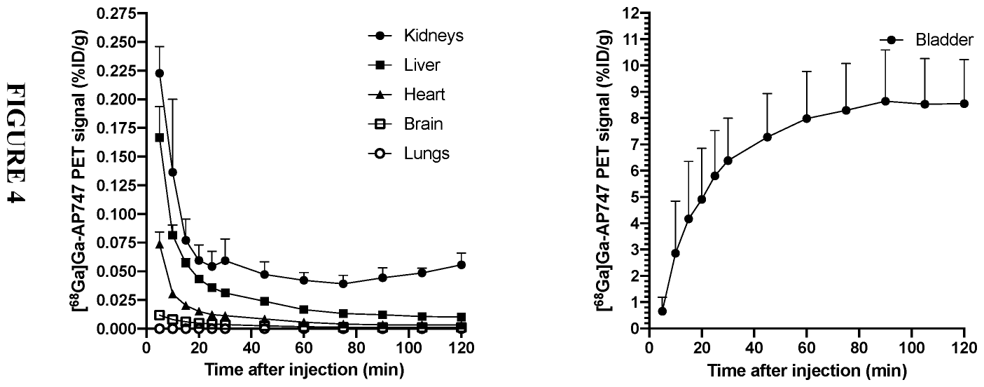

Because T84 has the higher expression level of APJ, a blocking strategy using autoradiography (FIG. 3) was performed on these cells. The remaining activity recorded by autoradiography in blocking condition was significantly reduced compared to classic conditions (P-value=0.0003). AP747 corresponds to NODAGA-Apelin13(F13A).

### Animal Experiments

### Biodistribution in Healthy Mice

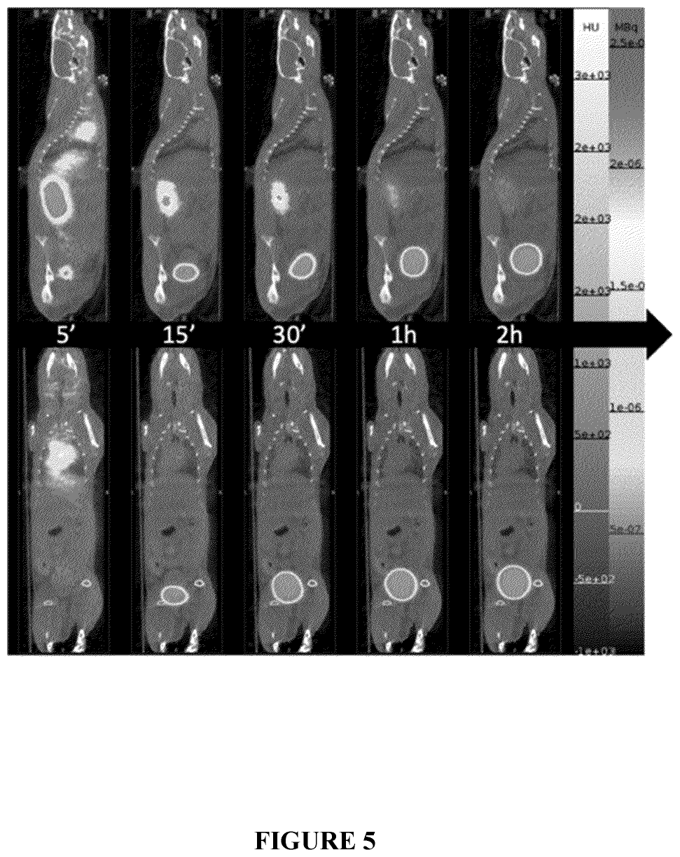

In healthy mice, PET signal quantification in organs (FIG. 4) highlights a suitable pharmacokinetic of [⁶⁸Ga]Ga-NODAGA-Apelin13(F13A) and a fast and mainly urinary excretion without noticeable accumulation in liver, lungs or brain. FIG. 5 shows representative PET biodistribution images of one mouse for two hours.

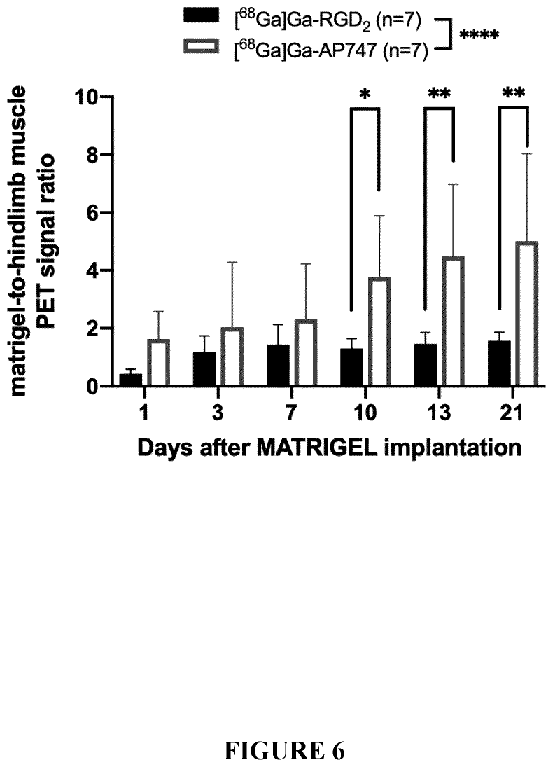

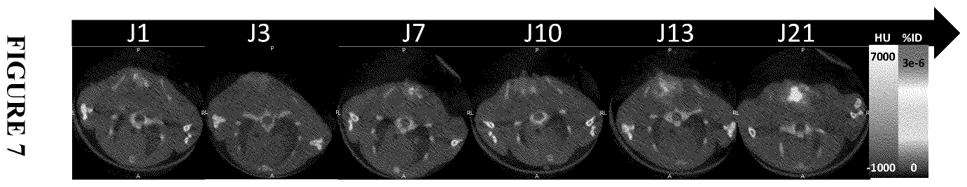

### Mouse Model of Hindlimb Ischemia and Matrigel

[⁶⁸Ga]Ga-NODAGA-Apelin13(F13A) accumulates in Matrigel plug and this accumulation is significantly higher than [⁶⁸Ga]Ga-NODAGA-RGD₂ on day 10 (P-value=0.0362), day 13 (P-value=0.0064) and day 21 (P-value=0.0016). [⁶⁸Ga]Ga-NODAGA-Apelin13(F13A) PET signal is all the more important over time (P-value=0.0051). Globally, over experiment period [⁶⁸Ga]Ga-NODAGA-Apelin13(F13A) PET signal is significantly superior to [⁶⁸Ga]Ga-NODAGA-RGD₂ PET signal (p=0.0000017) (FIG. 6). Corresponding PET images are shown on FIG. 7.

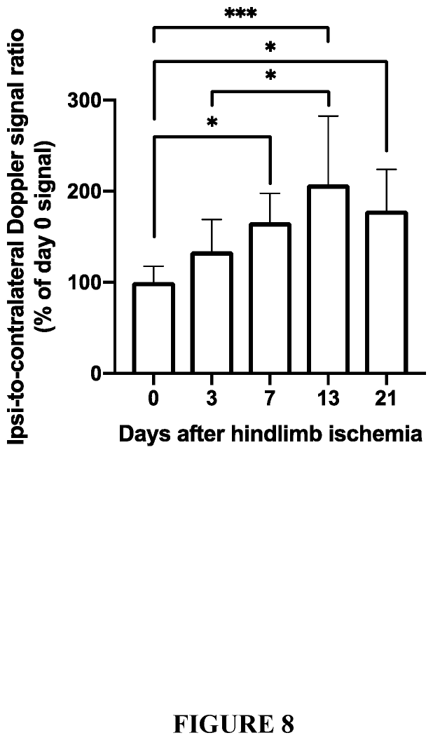

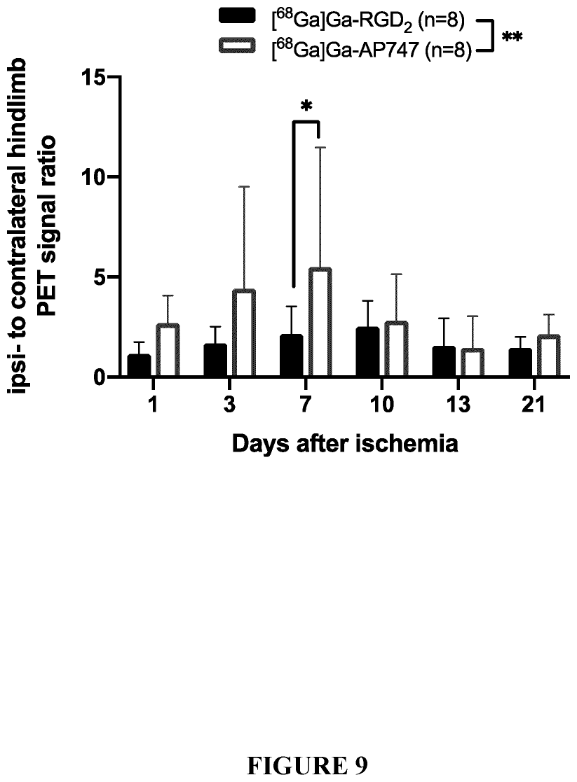

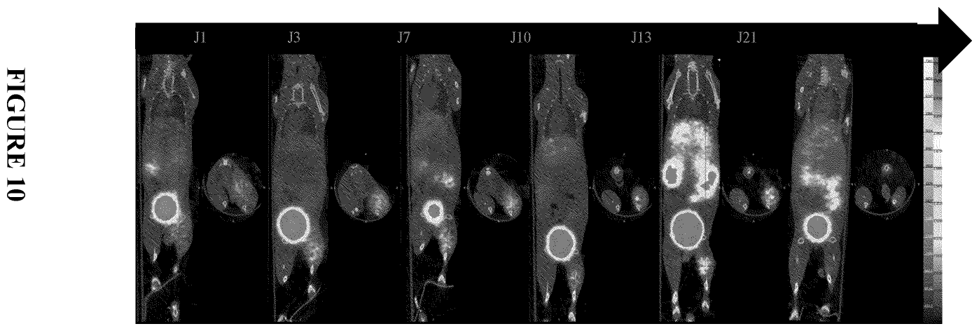

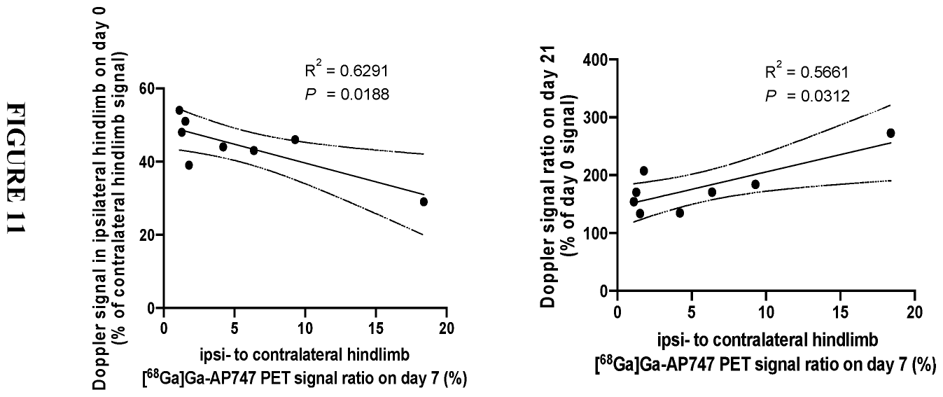

Ischemia-reperfusion monitoring of ischemic limb by LASER-Doppler (FIG. 8) was compared with non-ischemic limb and with perfusion after surgery (Day 1). In ischemic limb [⁶⁸Ga]Ga-NODAGA-Apelin13(F13A) PET signal (peak at Day 7) is notably better (P-value=0.0497), than [68Ga]Ga-NODAGA-RGD₂ PET signal (peak at Day 10) (FIG. 9). Corresponding PET images are represented on FIG. 10.

[⁶⁸Ga]Ga-NODAGA-Apelin13(F13A) PET signal in ischemic limb (corrected with non-ischemic limb) is significantly negatively correlated to LASER-Doppler signal at day of surgery (P two-tailed=0.0188). This means that [⁶⁸Ga]Ga-NODAGA-Apelin13(F13A) PET signal in ischemic limb is more important when ischemia injury is severe and so the hindlimb perfusion is low (FIG. 11).

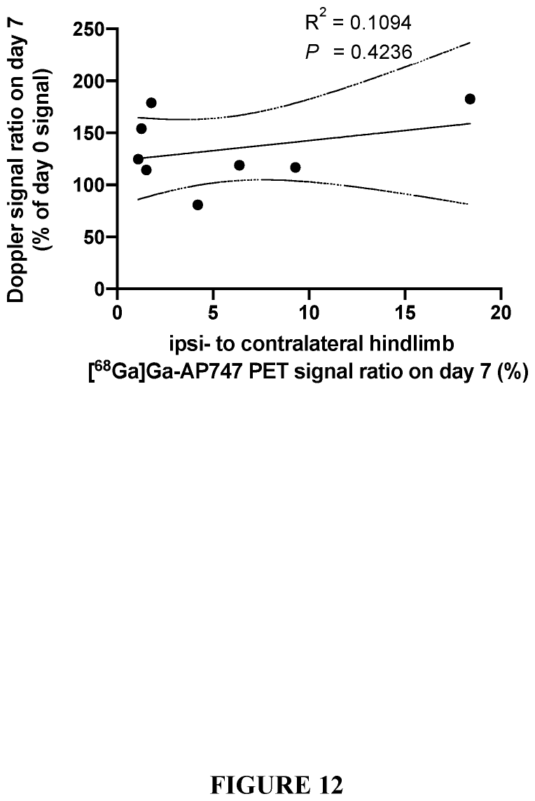

Another correlation was established. Indeed, [⁶⁸Ga]Ga-NODAGA-Apelin13(F13A) PET signal in ischemic limb (corrected with non-ischemic limb) is more important when reperfusion is longer (LASER-Doppler Day 21/Day 0) (P two-tailed=0.0196) (FIG. 11).

[⁶⁸Ga]Ga-NODAGA-Apelin13(F13A) PET signal in ischemic limb (corrected with non-ischemic limb) is not linked to day-input function (P two-tailed=0.4236) (FIG. 12).

### Ectopic Mouse Model of Human Colon Adenocarcinoma

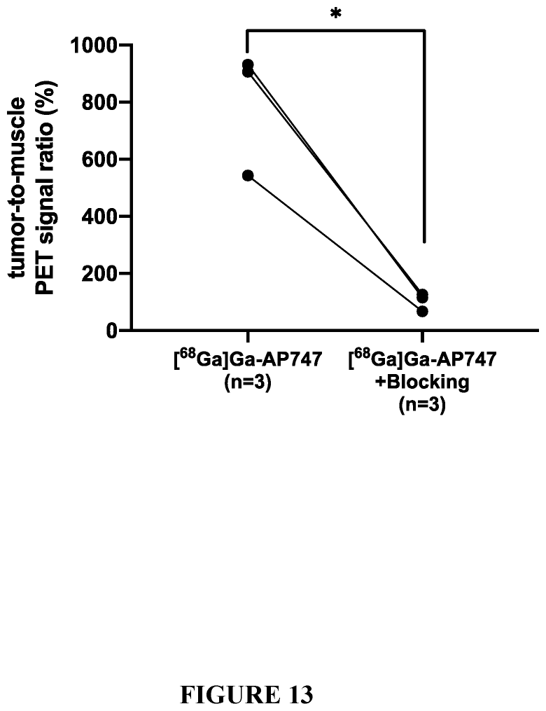

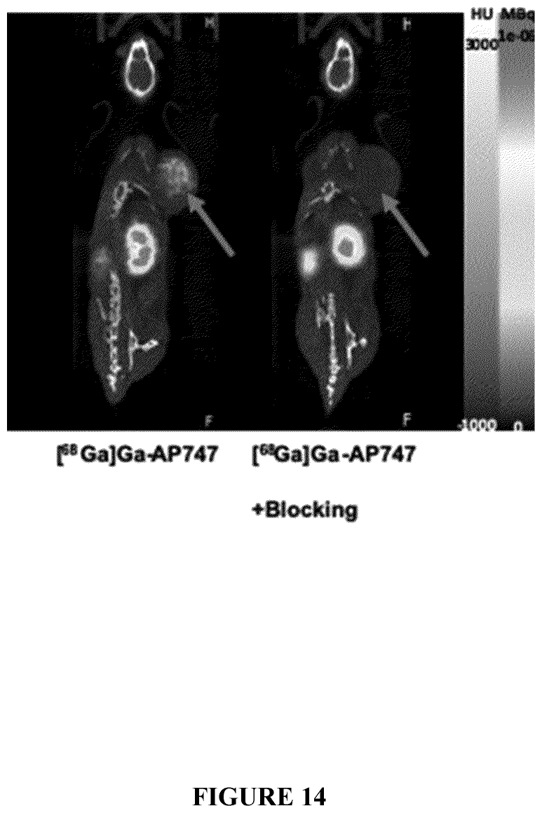

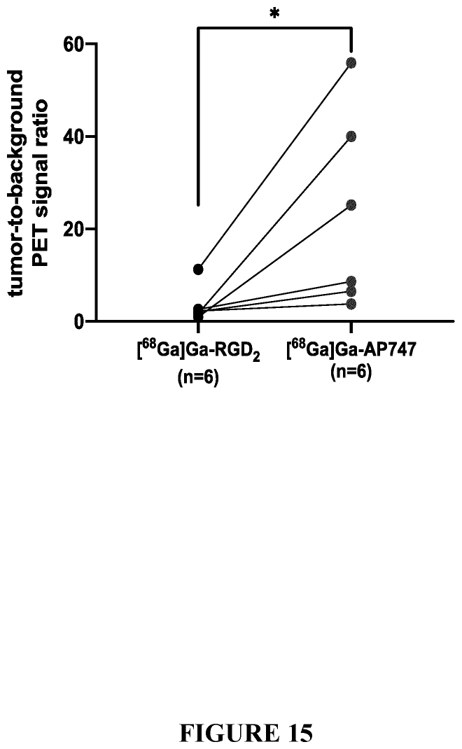

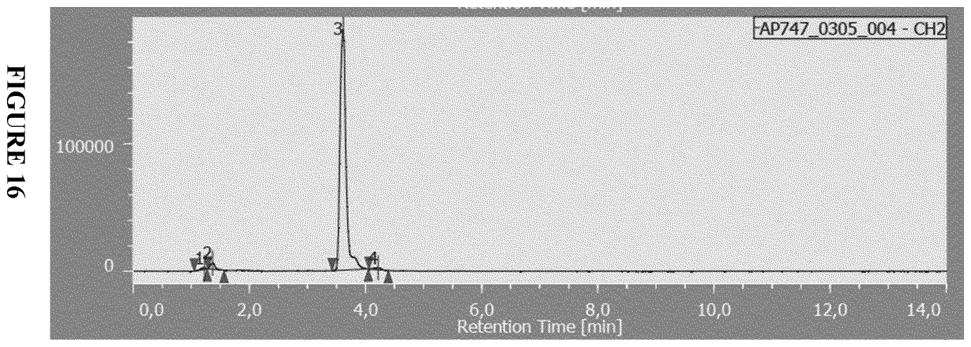

PET signal during in vivo blocking experiment on ectopic mouse model of colon adenocarcinoma was quantified and compared to classic conditions. Indeed, in classic conditions [⁶⁸Ga]Ga-NODAGA-Apelin13(F13A) PET signal level is significantly higher (793.3%±217.2) than [⁶⁸Ga]Ga-NODAGA-Apelin13(F13A) PET signal in blocking conditions (102.6%±31.37) (P-value=0.0235) (FIG. 13). Corresponding PET images are represented on FIG. 14.

[⁶⁸Ga]Ga-NODAGA-Apelin13(F13A) PET signal was compared to [⁶⁸Ga]Ga-NODAGA-RGD₂ PET signal (FIG. 15). PET signal quantification was corrected by background signal. [⁶⁸Ga]Ga-NODAGA-Apelin13(F13A) PET signal (23.33%21.09) was significantly (P-value=0.0480) higher than that of [⁶⁸Ga]Ga-NODAGA-RGD₂ (3.496%±3.841).

### CONCLUSIONS

Inventors herein describe the first radiotracer for PET imaging of APJ (herein identified as “AP747”). They first developed AP747 for detecting, imaging, measuring and/or monitoring APJ expressing-tissue. The radiomarker is usable as a companion tool for modulating therapeutic strategy, and as a tool to evaluate tissue angiogenesis.

After compound synthesis and its radiolabelling, AP747 targeting against APJ was validated thanks to blocking strategies monitored by autoradiography on cells expressing APJ. Once stability of the tracer had been validated, in vivo evaluation in PET imaging was performed.

Pharmacokinetic profile in rodent revealed a fast-urinary excretion without hepatic accumulation; an ideal profile for PET imaging agents and theragnostic approaches. Biodistribution evaluation on two well-described and characterized angiogenesis models: Matrigel (simple hypoxic model) and ischemic model (Hindlimb ischemia). In both models, the results showed that the PET signal of [68Ga]Ga-NODAGA-Apelin13(F13A) in angiogenesis outbreaks superior than [⁶⁸Ga]Ga-NODAGA-RGD₂ PET signal: gold standard in angiogenesis molecular imaging (Azizi Y et al., 2013).

Specificity of AP747 PET imaging signal was demonstrated through in vivo blocking experiments (pre-incubation to saturate binding sites of the tracer and appreciate the specificity of [⁶⁸Ga]Ga-NODAGA-Apelin13(F13A) with Apelin13(F13A)) on angiogenic and tumoral models, overexpressing APJ. Partial blocking (about 50%) can be explained by the small amount of cold Apelin13(F13A) used because of bad tolerance due to cardiovascular effects of Apelin as previously reported in literature (Benton G et al., 2014).

Targeting should be reflective of APJ overexpression in hypoxic conditions reported in literature and observed by Western-Blot during HUVEC TNF-stimulation. During angiogenesis sprouting, endothelial cell sprouts growing is known to be VEGF-guided, but other signals (repellent or attractive) can be useful in vessel formation moves. Massive secretion of apelin by endothelial tip cells promotes APJ expression by stalk cells, as well as their proliferation. Lumen formation in stalk cells involves vacuoles fusion and other mechanisms not fully explained, but APJ/Apelin system plays a major role during sprouting, as observed in Apelin-KO animals. Adhesive or repellent interactions between tip cells regulate sprouts and vessels fusion. Activated HUVEC fixation intensity appreciated by autoradiography was correlated to in vivo observations. APJ expression of gastrocnemius muscles of ischemic animals quantified by histology permits to evaluate links between PET signal intensity and APJ tissue expression. PET signal kinetic profile allows to assess APJ expression kinetic in hypoxic conditions (Matrigel and ischemic models). This expression seems to be intense and extended. Profiles observed on Matrigel and ischemic models are different:
In Matrigel model, signal is increasing, probably related to avascular and acellular contents of Matrigel, a minimum of 10 days is required to observe new vessels formation, and some supplementary days for obtain functional vessels (Chapman et al., 2014);
In ischemic model, a peak is observed at day 7 followed by an intensity diminution, probably linked to vascular development from popliteal anastomosis (Gronman, M et al., 2017);

Moreover, on ischemic model, LASER-Doppler signal at day 7 (peak) is correlated to late reperfusion index (Day 21/Day 1). In this way, [⁶⁸Ga]Ga-NODAGA-Apelin13(F13A) seems to be an early predictive factor of tissue perfusion, further argument supporting [⁶⁸Ga]Ga-NODAGA-Apelin13(F13A) as a tracer for evaluating tissue angiogenesis intensity.

In comparison with [⁶⁸Ga]Ga-NODAGA-RGD₂, [⁶⁸Ga]Ga-NODAGA-Apelin13(F13A) imaging appeared pertinent at an earlier stage, with stronger signal and that lingers longer. Information potency of [⁶⁸Ga]Ga-NODAGA-Apelin13(F13A) appears to be much more powerful than [⁶⁸Ga]Ga-NODAGA-RGD₂, actually in clinical development. Especially, regarding therapeutic and protector properties of Apelin, alone or as adjuvant, in ischemic pathologies comprising hind limb ischemia (Hasan, J et al., 2004), PET imaging that could evaluate in vivo expression or overexpression of APJ is usable as a tool to determine therapeutic eligibility, to monitor therapeutic efficiency, as prognostic or diagnostic index, like other theragnostic couples in clinical development or trials (Jakobsson, L et al., 2010).

Results observed in colon adenocarcinoma murine model with high level of [⁶⁸Ga]Ga-NODAGA-Apelin13(F13A) fixation, whose specificity had been checked by blocking strategy, are very relevant.

### Example 2

### Materials and Methods

### ⁶⁷Ga-Radiolabelling

[⁶⁷Ga]Ga-citrate (200 MBq, CURIUM) was converted in [⁶⁷Ga]GaCl₃ using two Light silica Sep-pac (Waters, refWAT023537). Briefly, [⁶⁷Ga]Ga-citrate was loaded on the cartridge and then eluted using 1 mL HCl 0.1M (Rottem, KT720P) in form of [⁶⁷Ga]GaCl₃ and subsequently used for radiolabelling as described for [⁶⁸Ga]GaCl₃. The final product was formulated in 3 mL PBS.

### Radiochemical Purity Control

Radio-UV-HPLC analyses were performed using a Phenomenex Luna C18 column (4 mL/min, λ=220 nm C18; 150 mm×4.6 mm×5 μm). HPLC conditions were: 0-2 min: 90% ACN (A), 10% water in 0.1% TFA (B), 2-10 min: 90%→10% A; 10%→90% B, 10-12 min: 10% A; 90% B, 12-14 min: 10%→90% A; 90%→10% B. The analytical HPLC system used was a JASCO system with ChromNAV software, a PU-2089 Plus quaternary gradient pump, a MD-2018 Plus photodiode array detector and Raytest Gabi Star detector. TLC analysis were also carried out (miniGITA plate reader, acquisition time of 1 min, Rf impurities≥0.8, Rf ⁶⁸Ga-bioconjugates ≤0.1 using citrate buffer pH5 as mobile phase and ITLC-SG as stationary phase.

### Hydrophilicity

Hydrophilicity of [⁶⁷Ga]Ga-AP747 was assessed by the water-octanol partition/distribution coefficient method. In a centrifuge tube, 500 μL of 1-octanol was added to 500 μL of phosphate-buffered saline (pH 7.4) containing the radiolabeled peptide (50 kBq). After equilibrium, the solution was vigorously stirred for 5 min at room temperature and subsequently centrifuged (4000 rpm, 5 min) to yield two immiscible layers. Aliquots of 100 μL were taken from each layer and the radioactivity in the samples was determined by a gamma counter (Perkin Elmer, Waltham, Mass., USA).

### Saturation Binding Assay.

The affinity of [⁶⁷Ga]Ga-AP747 was studied on T84 cells seeded at a density of 250.10³ cells per well in 24-well plates (Corning®) and incubated overnight with complete medium. Well plates were first set on ice 30 minutes before the beginning of the experiment. [⁶⁷Ga]Ga-AP747 was then added to the medium at concentration of (0.1, 1, 10, 100, 250 nM) and cells were incubated (in quadruplicates) for 2 hours at 4° C. Incubation was stopped by removing medium and washing cells twice with ice-cold PBS. Finally, cells were treated with NaOH (1M) and radioactivity was measured in a gamma counter. In order to assess for non-specific affinity, excess non-radioactive apelin-13 (final concentration 1 M), was added to selected wells.

### In Vitro Internalization and Efflux Studied Studies.

T84 cells were cultured as described in saturation binding experiments above.

For internalization studies, 50 kBq of [⁶⁷Ga]Ga-AP747 were added to the medium the day of the experiments and the cells were incubated (in quadruplicates) during 10, 30 or 60 minutes at 37° C. Three minutes before the end of the incubation time, internalization was stopped on ice and the supernatant was removed. Internalization was then stopped by eliminating the supernatant and each well was washed with 3×250 μL of ice-cold PBS. The membrane-bound fraction was retrieved in 2×250 μL sodium acetate buffer (20 mM, pH 5) for 5 min. Finally, cells were treated with 500 μL of NaOH (1 M). The radioactivity of the membrane-bound fraction and the internalized fraction was measured in a gamma counter. The experiment was performed twice. To also verify receptor specificity, blocking experiments were performed by using 1 μM of apelin-13.

For efflux experiments, 10 kBq of [⁶⁷Ga]Ga-AP747 were added to the medium the day of the experiments and the cells were incubated (in octoplicates) for 30 minutes at 37° C. Three minutes before the end of the incubation time, internalization was stopped on ice and the supernatant was removed. Each well was washed with 1 mL of ice-cold PBS. The membrane-bound fraction was retrieved in 2 mL sodium acetate buffer (20 mM, pH 5) for 2 min, each well was rinsed a second time with 1 mL ice-cold PBS and fresh culture medium was added. At each time point (10, 30, 60 and 120 minutes), the efflux was stopped by collecting the medium and washing cells twice with ice-cold PBS. Finally, cells were treated with NaOH (1 M). The radioactivity of the collected culture medium supernatant, the PBS wash fractions, and the total internalized fraction was measured in a gamma counter. The experiment was performed twice.

### Radiosynthesis of Al[¹⁸F]F-NODA-Apelin-F13A

Aluminium chloride (AlCl₃·6H₂O), sodium acetate (NaOAc), potassium hydrogenocarbonate (KHCO₃), glacial acetic acid (AcOH), water for HPLC, acetonitrile for HPLC, trifluoroacetic acid and pH paper were purchased from Sigma (France). The analytic reverse phase HPLC column (Luna C18 150×4.6 mm 5 μm) was purchased from Phenomenex (France). Solid-phase extraction cartridge (Sep-Pak QMA light) was purchased from Waters (France). No carrier-added [¹⁸F] fluoride was trapped on the anion-exchange resin. The cartridge was washed with 5 mL of HPLC water. The cartridge carrying the ¹⁸F-anions was eluted with 600 μL of a 0.4 M solution of KHCO₃. A pH of 4.5 required for ¹⁸F-chelation by addition of glacial acetic acid was obtained. The pH value was determined using pH-paper. 50 μL of the ¹⁸F-solution were incubated 10 minutes at room temperature with 3 μL of a 2 mM solution of AlCl₃·6H₂O. Then, 9 μL of NODA-Apelin (2 mM in 0.5 M of NaOAc) were added to the previous reaction mixture. The solution was incubated at 100° C. for 10 minutes. 20 μL of the reaction mixture was injected through HPLC as described upper.

### Radiosynthesis of [⁶⁸Ga]Ga-DOTA-Apelin-F13A

70 μL of 1 mol·L⁻¹ sodium acetate trihydrate solution were added to 4 μg/10 μL of DOTA-Apelin-F13A. 500p of freshly eluted [⁶⁸Ga]GaCl₃ were added to the reactor. The mixture was heated at 110° C. for 10 min, then allowed to cool at room temperature for 5 min. A tC18-light cartridge was preconditioned with 1 mL of 90% ethanol, then 2 mL of HPLC water, and loaded with the reaction product. The cartridge was washed with 2 mL of HPLC water. Finally, [⁶⁸Ga]Ga-DOTA-Apelin-F13A was eluted from the tC18 cartridge with 0.5 mL of 50% ethanol solution in 0.9% NaCl. Radiochemical purity was checked before and after purification by radio-thin layer chromatography (radioTLC) on ITLC/sg paper with 0.1M sodium citrate solution pH=5 ([⁶⁸Ga]Ga-DOTA-Apelin-F13A at Rf0; [⁶⁸Ga]Ga³⁺ at the front, Rf1).

### Orthotopic Mouse Model of Human Glioblastoma

Human glioblastoma xenografts were achieved by orthotopic injections 5×10⁵ U87 cells (3 μL, PBS+/+) into 8-week-old female athymic nude mice (Charles River) under 2% isoflurane anesthesia. Stereotaxic injections using a Hamilton microsyringe were realized in left striatum (coordinates: −2 mm dorsal/ventral, +1 mm lateral, and +1 mm anterior/posterior from bregma). Mice were allowed for resting for 4 weeks.

[⁶⁸Ga]Ga-AP747 microPET CT of Orthotopic Mouse Model of Human Glioblastoma

Mice bearing orthotopic human glioblastoma (n=3) were IV injected with [⁶⁸Ga]Ga-RGD₂ (1.99±0.25 MBq) or with [⁶⁸Ga]Ga-AP747 (2.95±0.15 MBq) under anesthesia. PET images were acquired 1 h after IV injection on a NanoPET/CT (Mediso) under 2% isoflurane anesthesia. Quantitative region-of-interest (ROI) analysis of the PET images was performed on attenuation- and decay-corrected PET images using VivoQuant software (InVicro) and tissue uptake values were presented as a left-to-right hemisphere activity ratio. Left-to-right hemisphere ratios were compared using the paired t-test.

Middle Cerebral Artery Occlusion (MCAO) Followed with Reperfusion in Rats

6-8-month-old female rats were intubated, and mechanically ventilated with 3.0 vol % sevoflurane in a gas mixture of 30% oxygen and 70% nitrogen. Focal cerebral ischemia was induced by occluding the right middle cerebral artery with a monofilament coated with silicone (diameter adjusted to the weight of the animal). After a 60-min ischemia, the filament was withdrawn allowing reperfusion. Rats were allowed for resting for 2 days.

[⁶⁸Ga]Ga-AP747 microPET CT of MCAO Rats

MCAO rats (n=3) were IV injected with [⁶⁸Ga]Ga-AP747 (12.05±0.75 MBq) under anesthesia the day before MCAO, right after MCAO, and everyday up to day 7 post-MCAO. MicroPET images were acquired for 20 min, 2 h after injection, on a NanoPET/CT (Mediso) under 2% isoflurane anesthesia. Quantitative region-of-interest (ROI) analysis of the PET images was performed on attenuation- and decay-corrected PET images using VivoQuant software (InVicro) and tissue uptake values were presented as the quantified ipsi- to contralateral microPET signal ratio and compared to the day before MCAO using a one-way ANOVA.

### Results

[⁶⁷Ga]Ga-AP747 and [⁶⁸Ga]Ga-AP747 were obtained with high radiochemical purity (>95%), high apparent molar activity >10 MBq/μg and high volumic activity >30 MBq/mL.

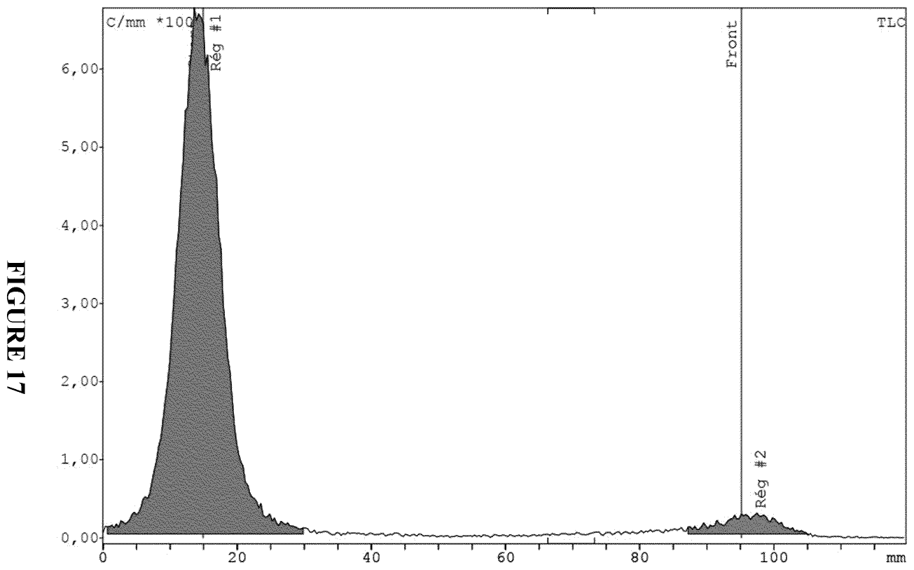

Representative radio-HPLC chromatogram of [⁶⁸Ga]Ga-AP747 is displayed in FIG. 16. Representative radio-TLC chromatogram of [⁶⁷Ga]Ga-AP747 is also displayed in FIG. 17.

### Hydrophilicity

[⁶⁷Ga]Ga-AP747 was found to be hydrophilic with a log D_{7.4} value of −3.03±0.02.

### Saturation Binding Assay

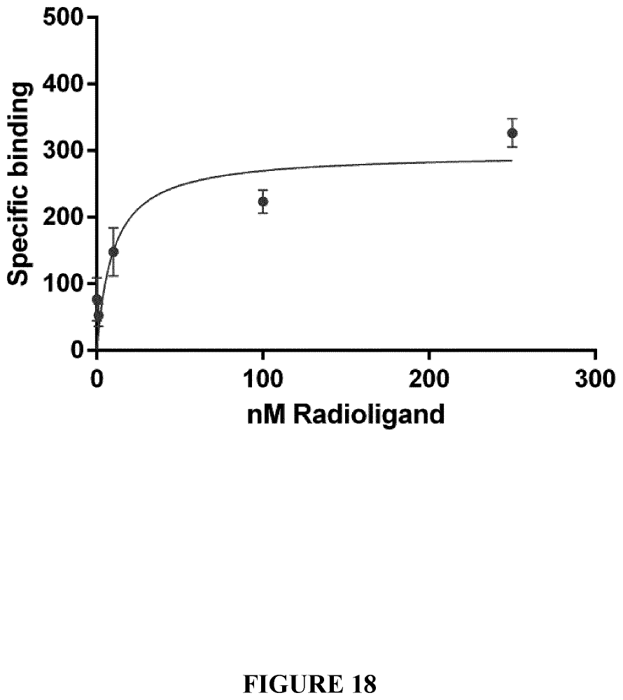

The specific receptor binding of [⁶⁷Ga]Ga-AP747 for APJ receptor was investigated on T84 cells. Saturation binding curves revealed nanomolar affinity was with a K_{d} value of 11.85±2.8 nM (FIG. 18).

### In Vitro Internalization and Efflux Studied Studies.

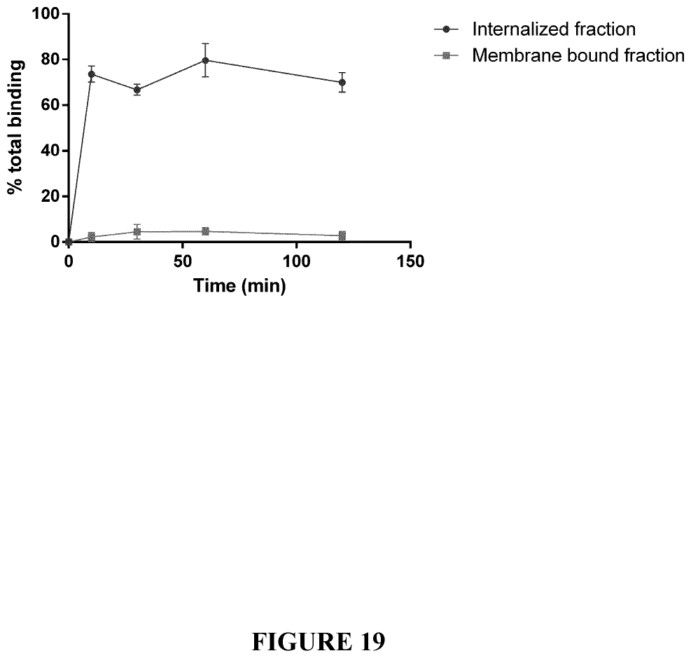

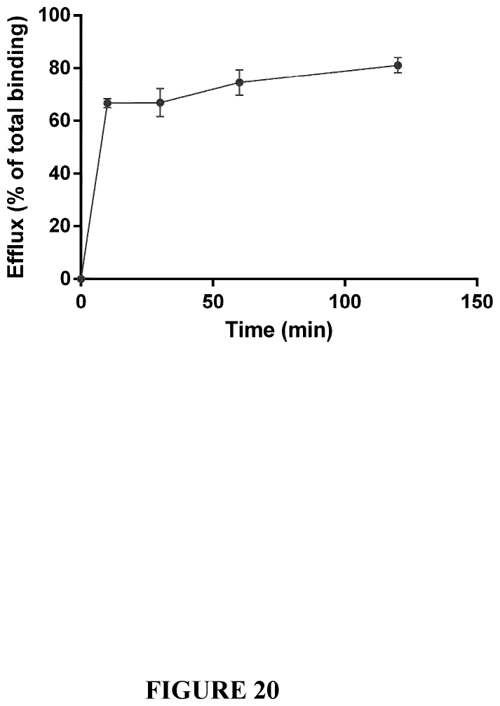

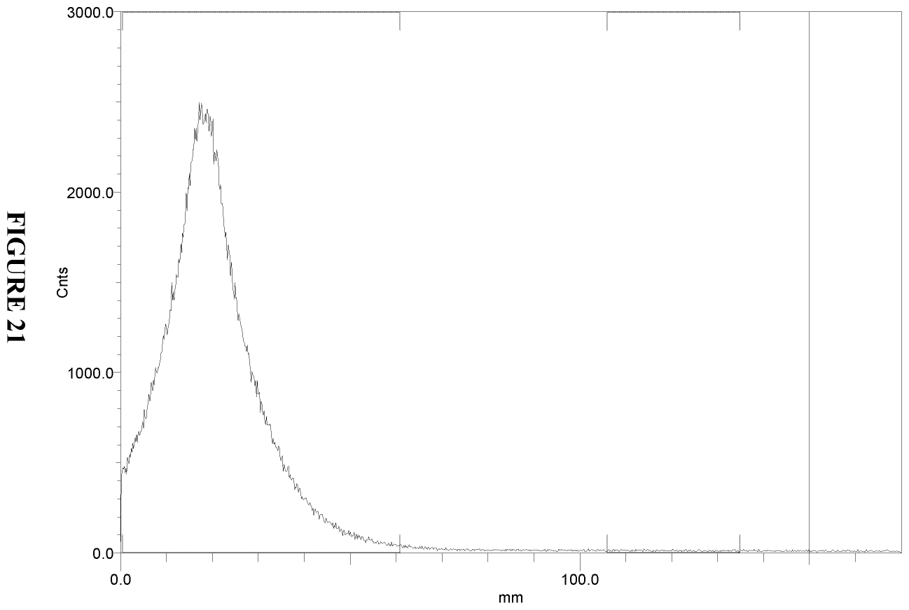

The APJ receptor-mediated internalization and the APJ receptor membrane-bound fraction of [⁶⁷Ga]Ga-AP747 into T84 cells were analyzed. Specific and time dependent internalization into T84 cells was observed with a maximum of 79.7±7.3% of the cell associated radioactivity being internalized at 60 min. The receptor specific and time dependent membrane-bound fraction of [⁶⁷Ga]Ga-AP747 was low (<5%) at any time point (FIG. 19)

[⁶⁷Ga]Ga-AP747 was further evaluated regarding cellular efflux on T84 cells. A high and fast efflux of internalized radioactivity was found for [⁶⁷Ga]Ga-AP747. Already 10 minutes post-internalization, 66.7±1.7% of the total binding was externalized. Efflux increased over time to reach 81.2±2.9% at 2 h (FIG. 20).

### Radiosynthesis of Al[¹⁸F]F-NODA-Apelin-F13A

NODA-Apelin-F13A was rapidly and successfully radiolabeled with [¹⁸F]F in 10 minutes at 100° C. by chelation via Al-bound ¹⁸F. The quality control assessed by HPLC, before purification, showed a labeling efficiency of 47%. Further purification of the Al[¹⁸F]F-NODA-Apelin was achieved through a C18 cartridge.

### Radiosynthesis of [⁶⁸Ga]Ga-DOTA-Apelin-F13A

DOTA-Apelin-F13A was rapidly and successfully radiolabeled with [⁶⁸Ga]Ga. The quality control assessed by TLC, before purification, showed a labeling efficiency of 76%. Further purification of the [⁶⁸Ga]Ga-DOTA-Apelin-F13A was achieved through a C18 cartridge reaching a ≥99% radiochemical purity (FIG. 21).

[⁶⁸Ga]Ga-AP747 microPET CT of Orthotopic Mouse Model of Human Glioblastoma

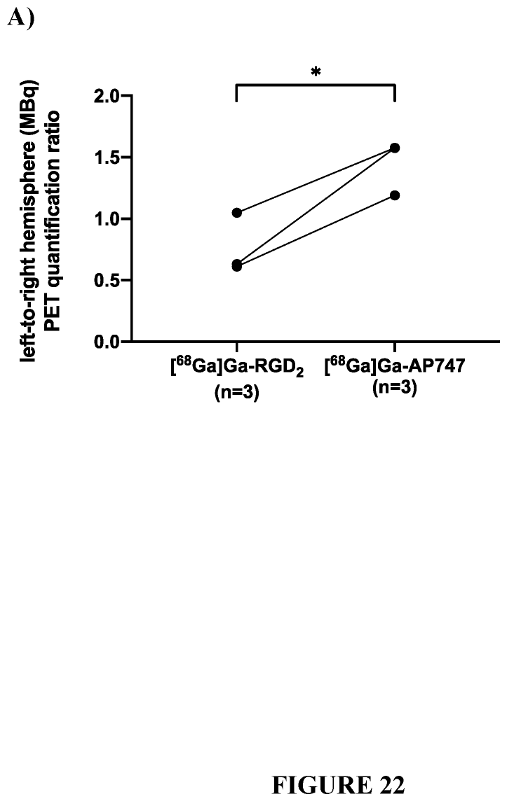

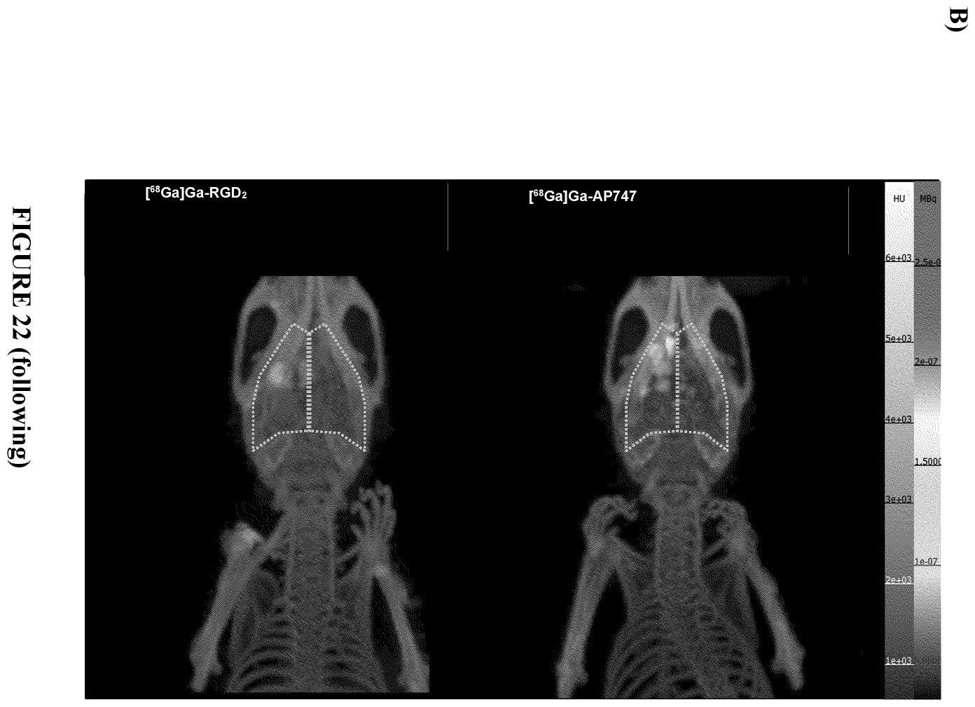

Ipsi-to-contralateral [⁶⁸Ga]Ga-AP747 microPET signal quantification ratio was significantly higher than that of [⁶⁸Ga]Ga-RGD₂ in the same glioblastoma mice (1.45±0.22 and 0.76±0.25 respectively, *P=0.0346, n=3, FIG. 22).

[⁶⁸Ga]Ga-AP747 microPET CT of MCAO Rats

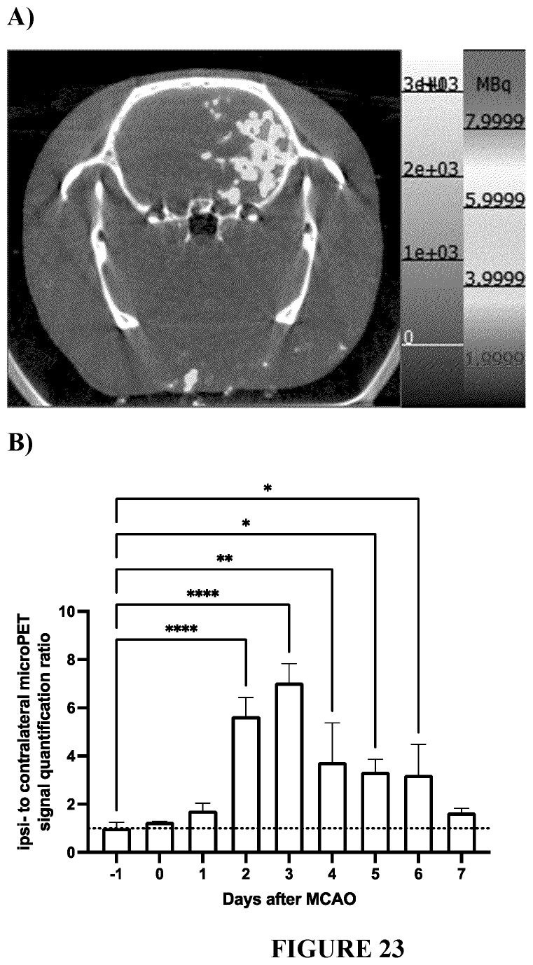

After no significant modification on the day of MCAO (1.26±0.03, n=3) and the day after (1.74±0.31, n=3), ipsi-to-contralateral [⁶⁸Ga]Ga-AP747 microPET signal quantification ratio significantly peaked on day 2 (5.65±0.77, ****P<0.0001, n=3) and day 3 (7.05±0.78, ****P<0.0001, n=3), then slightly decreased on day 4 (3.75±1.62, **P=0.0029, n=3), day 5 (3.34±0.52, *P=0.0121, n=3) and day 6 (3.21±1.27, *P=0.0185, n=3) until back to baseline on day 7 (1.65±0.17, n=3, FIG. 23).

### CONCLUSIONS

Altogether, inventors herein demonstrate that:

[⁶⁸Ga]Ga-AP747 (i.e. [⁶⁸Ga]Ga-NODAGA-Apelin) is a powerful radiotracer useful for labelling and/or imaging glioblastoma in vivo as shown in micro PET/CT of orthotopic mouse model of human glioblastoma (FIG. 22). In comparison with [⁶⁸Ga]Ga-NODAGA-RGD₂, [⁶⁸Ga]Ga-AP747 imaging appeared with stronger signal. Information potency of [⁶⁸Ga]Ga-AP747 appears to be much more powerful than [⁶⁸Ga]Ga-NODAGA-RGD₂.

The present results demonstrate that [⁶⁸Ga]Ga-AP747 is suitable for use as a Positron Emission Tomography (PET-CT) radiotracer and is suitable for use for labelling and/or imaging in vivo or ex vivo a tissue or organ expressing the APJ receptor or for use in vivo for detecting, measuring, diagnosing, staging and/or monitoring a cancer.

[⁶⁸Ga]Ga-AP747 is also a powerful radiotracer for quantifying APJ receptor expression kinetics following ischemia as shown on a rat model of middle cerebral artery occlusion (MCAO) followed with reperfusion (FIG. 23).

Inventors successfully generated the tracers [⁶⁸Ga]Ga-DOTA-Apelin and Al¹⁸F-NODA-Apelin with a an excellent radiochemical purity greater than 99%.

They successfully developed the tracer [⁶⁷Ga]Ga-NODAGA-Apelin with high radiochemical purity (>95%) for detecting, imaging, measuring and/or monitoring APJ expressing-tissue. After compound synthesis and its radiolabelling, [⁶⁷Ga]Ga-NODAGA-Apelin binding on carcinoma cells (T84) was studied and revealed a great (nanomolar) affinity of [⁶⁷Ga]Ga-NODAGA-Apelin for APJ receptor on T84 cells. Likewise, inventors showed the APJ receptor-mediated internationalization as well as the high and fast efflux for [⁶⁷Ga]Ga-NODAGA-Apelin. As switching from [⁶⁸Ga]Ga to [⁶⁷Ga]Ga has no influence on the chemical structure of the radiotracer, these in vitro results obtained with [⁶⁷Ga]Ga-AP747 can be extrapolated to [⁶⁸Ga]Ga-AP747. Similarly, the in vivo results obtained with [68Ga]Ga-AP747 can be extrapolated to [⁶⁷Ga]Ga-AP747. Altogether, the results show that [⁶⁸Ga]Ga-AP747 and [⁶⁷Ga]Ga-AP747 are suitable for use as PET-CT radiotracers.

### REFERENCES

Activation of Catalase by Apelin Prevents Oxidative Stress-linked Cardiac Hypertrophy—Foussal—2010—FEBS Letters—Wiley Online Library.” n.d. Accessed Aug. 19, 2019.
Azizi Y, Faghihi M, Imani A, Roghani M, Nazari A. Post-infarct treatment with [Pyr1]-apelin-13 reduces myocardial damage through reduction of oxidative injury and nitric oxide enhancement in the rat model of myocardial infarction. Peptides. 2013; 46:76-82.
Benton, G., Arnaoutova, I., George, J., Kleinman, H. K. & Koblinski, J. Matrigel: From discovery and ECM mimicry to assays and models for cancer research. Advanced Drug Delivery Reviews. 2014; 79-80, 3-18.
Berta J, Kenessey I, Dobos J, et al. Apelin Expression in Human Non-small Cell Lung Cancer: Role in Angiogenesis and Prognosis. Journal of Thoracic Oncology. 2010; 5(8):1120-1129.
Brame A L, Maguire J J, Yang P, et al. Design, Characterization, and First-In-Human Study of the Vascular Actions of a Novel Biased Apelin Receptor Agonist. Hypertension. 2015; 65(4):834-840.
Carroll A O, Lolait S J, Harris L E, Pope G R. The apelin receptor APJ: journey from an orphan to a multifaceted regulator of homeostasis. 1998.
Cesirio J M S, Brito R B O, Malta C S, et al. A simple method to induce hypoxia-induced vascular endothelial growth factor-A (VEGF-A) expression in T24 human bladder cancer cells. In Vitro CellDevBiol-Animal. 2017; 53(3):272-276.
Chapman, N. A., Dupré, D. J. & Rainey, J. K. The apelin receptor: Physiology, pathology, cell signalling, and ligand modulation of a peptide-activated class A GPCR. Biochem Cell Biol 92. 2014; 431-440.
Chen T, Liu N, Xu G-M, Liu T-J, Liu Y, Zhou Y, Huo S-B, Zhang K (2017) Apelin13/APJ promotes proliferation of colon carcinoma by activating Notch3 signaling pathway. Oncotarget 8:101697-101706.
Dowda B F O, Heiberb M, Chanb A, et al. A human gene that shows identity with the gene encoding the angiotensin receptor is located on chromosome 11. 1993; 136:355-360.
Dray C, Knauf C, Valet P, Masri B. Apelin receptors: From signaling to antidiabetic strategy. Eur J Pharmacol. 2015:1-11.
Eyries Melanie, Siegfried Géraldine, Ciumas Mariana, et al. Hypoxia-Induced Apelin Expression Regulates Endothelial Cell Proliferation and Regenerative Angiogenesis. Circulation Research. 2008; 103(4):432-440.
Foldes G, Horkay F, Szokodi I, et al. Circulating and cardiac levels of apelin, the novel ligand of the orphan receptor APJ, in patients with heart failure. Biochemical and Biophysical Research Communications. 2003; 308(3):480-485.
Gerbier R, Leroux V, Couvineau P, Alvear-perez R. New structural insights into the apelin receptor: identification of key residues for apelin binding. 2019.
Gronman, M. et al. Imaging of αvβ3 integrin expression in experimental myocardial ischemia with [68Ga]NODAGA-RGD positron emission tomography. Journal of Translational Medicine. 2017; 15, 144.
Hall C, Ehrlich L, Venter J, O'Brien A, White T, Zhou T, et al. Inhibition of the apelin/apelin receptor axis decreases cholangiocarcinoma growth. Cancer Lett. 1 févr 2017; 386:179-88.
Harford-Wright E, Andre-Gregoire G, Jacobs K A, et al. Pharmacological targeting of apelin impairs glioblastoma growth. Brain. 2017; 140(11):2939-2954.
Hasan, J. et al. Quantitative Angiogenesis Assays in vivo—A Review. Angiogenesis. 2004; 7, 1-16.
Heo K, Kim Y H, Sung H J, et al. Hypoxia-induced up-regulation of apelin is associated with a poor prognosis in oral squamous cell carcinoma patients. Oral Oncology. 2012; 48(6):500-506.
Hou J, Zhong T, Guo T, et al. Apelin promotes mesenchymal stem cells survival and vascularization under hypoxic-ischemic condition in vitro involving the upregulation of vascular endothelial growth factor. Experimental and Molecular Pathology. 2017; 102(2):203-209.
Iturrioz X, Gerbier R, Leroux V, Alvear-Perez R, Maigret B, Llorens-Cortes C. By Interacting with the C-terminal Phe of Apelin, Phe255 and Trp259 in Helix VI of the Apelin Receptor Are Critical for Internalization. J Biol Chem. 2010; 285(42):32627-32637.
Jakobsson, L. et al. Endothelial cells dynamically compete for the tip cell position during angiogenic sprouting. Nature Cell Biology. 2010; 12, 943-953.
Japp A G, Cruden N L, Barnes G, et al. Acute Cardiovascular Effects of Apelin in Humans: Potential Role in Patients With Chronic Heart Failure. Circulation. 2010; 121(16):1818-1827.
Kawamata Y, Habata Y, Fukusumi S, et al. Molecular properties of apelin: tissue distribution and receptor binding. 2001; 1538:162-171.
Kasai A, Ishimaru Y, Kinjo T, et al. Apelin Is a Crucial Factor for Hypoxia-Induced Retinal Angiogenesis. ATVB. 2010; 30(11):2182-2187.
Kasai A, Ishimaru Y, Higashino K, et al. Inhibition of apelin expression switches endothelial cells from proliferative to mature state in pathological retinal angiogenesis. Angiogenesis. 2013; 16(3):723-734.
Khan P, Maloney P R, Hedrick M, et al. Functional Agonists of the Apelin (APJ) Receptor. In: Probe Reports from the NIH Molecular Libraries Program. Bethesda (Md.): National Center for Biotechnology Information (US); 2010.
Kleinz M J, Davenport A P. Immunocytochemical localization of the endogenous vasoactive peptide apelin to human vascular and endocardial endothelial cells. 2004; 118:119-125.
Langelaan D N, Reddy T, Banks A W, Dellaire G, Dupré DJ, Rainey J K. Biochimica et Biophysica Acta Structural features of the apelin receptor N-terminal tail and first transmembrane segment implicated in ligand binding and receptor trafficking. BBA—Biomembr. 2013; 1828:1471-1483.
Le Gonidec S, Chaves-Almagro C, Bai Y, et al. Protamine is an antagonist of apelin receptor, and its activity is reversed by heparin. The FASEB Journal. 2017; 31(6):2507-2519.
Li F Y, Li L, Qin X, et al. Apelin-induced vascular smooth muscle cell proliferation: the regulation of cyclin D1. Frontiers in bioscience: a journal and virtual library. 2008; 13:3786-3792.
Li X, Zhang X, Li F, et al. 14-3-3 mediates apelin-13-induced enhancement of adhesion of monocytes to human umbilical vein endothelial cells. Acta Biochim Biophys Sin (Shanghai). 2010; 42(6):403-409.
Macaluso N J M, Pitkin S L, Maguire J J, Davenport A P, Glen R C. Discovery of a Competitive Apelin Receptor (APJ) Antagonist. ChemMedChem. 2011; 6(6):1017-1023.
Maguire J J, Kleinz M J, Pitkin S L, Davenport A P. Isoform in the Human Heart Vasoactive Mechanisms and Inotropic Action in Disease. 2009:598-604.
Maloney P R, Khan P, Hedrick M, et al. Discovery of 4-oxo-6-((pyrimidin-2-ylthio)methyl)-4H-pyran-3-yl 4-nitrobenzoate (ML221) as a functional antagonist of the apelin (APJ) receptor. Bioorg Med Chem Lett. 2012; 22(21):6656-6660.
Mota N De, Goazigo A R, Messari S El, et al. Apelin, a potent diuretic neuropeptide counteracting vasopressin actions through inhibition of vasopressin neuron activity and vasopressin release. 2004; 101.
Muto J, Shirabe K, Yoshizumi T, et al. The Apelin-APJ System Induces Tumor Arteriogenesis in Hepatocellular Carcinoma. Anticancer Res. 2014; 34(10):5313-5320.
Picault F-X, Chaves-Almagro C, Projetti F, Prats H, Masri B, Audigier Y. Tumour co-expression of apelin and its receptor is the basis of an autocrine loop involved in the growth of colon adenocarcinomas. Eur J Cancer. 1 févr 2014; 50(3):663-74.
Schreiber C A, Holditch S J, Ikeda A G and Y. Sustained ELABELA Gene Therapy in High-salt Diet-induced Hypertensive Rats [Internet]. Vol. 16, Current Gene Therapy. 2016 [cité 5 mai 2020]. p. 349-60. Disponible sur: http://www.eurekaselect.com/147524/article
Tatemoto K, Hosoya M, Habata Y, et al. Isolation and Characterization of a Novel Endogenous Peptide Ligand for the Human APJ Receptor. 1998; 476:471-476.
Trifonov L, Afri M, Palczewski K, Gruzman* EEK and A. An Expedient Synthesis of CMF-019: (S)-5-Methyl-3-{1-(pentan-3-yl)-2-(thiophen-2-ylmethyl)-1H-benzo[d]imidazole-5-carboxamido}hexanoic Acid, a Potent Apelin Receptor (APJ) Agonist. Medicinal Chemistry. Published Oct. 31, 2018.
Liu M, Li H, Zhou Q, et al. ROS-Autophagy pathway mediates monocytes-human umbilical vein endothelial cells adhesion induced by apelin-13. Journal of Cellular Physiology. 2018; 233(10):6839-6850.
Li Y, Bai Y, Jiang Y, et al. Apelin-13 Is an Early Promoter of Cytoskeleton and Tight Junction in Diabetic Macular Edema via PI-3K/Akt and MAPK/Erk Signaling Pathways. BioMed Research International. 2018.
Picault F-X, Chaves-Almagro C, Projetti F, Prats H, Masri B, Audigier Y. Tumour co-expression of apelin and its receptor is the basis of an autocrine loop involved in the growth of colon adenocarcinomas. Eur J Cancer. 1 févr 2014; 50(3):663-74.
Podgórska M, Diakowska D, Pietraszek-Gremplewicz K, Nienartowicz M, Nowak D. Evaluation of Apelin and Apelin Receptor Level in the Primary Tumor and Serum of Colorectal Cancer Patients. J Clin Med [Internet]. 20 sept 2019 [cité 29 Jan. 2020]; 8(10).
Wang M, Gupta R C, Rastogi S, et al. Effects of acute intravenous infusion of apelin on left ventricular function in dogs with advanced heart failure. J Card Fail. 2013; 19(7):509-516.
Wan Y, Zeng Z, Xi M, et al. Dysregulated microRNA-224/apelin axis associated with aggressive progression and poor prognosis in patients with prostate cancer. Human Pathology. 2015; 46(2):295-303.
Wysocka M B, Pietraszek-gremplewicz K, Nowak D. The Role of Apelin in Cardiovascular Diseases, Obesity and Cancer. 2018; 9:1-15.
Wysocka M B, Pietraszek-Gremplewicz K, Nowak D. The Role of Apelin in Cardiovascular Diseases, Obesity and Cancer. Front Physiol. 2018; 9.
Xin Q, Cheng B, Pan Y, Liu H, yang C, Chen J, et al. Neuroprotective effects of apelin-13 on experimental ischemic stroke through suppression of inflammation. Peptides. 1 Jan. 2015; 63:55-62.
Yeganeh-Hajahmadi M, Najafipour H, Rostamzadeh F. The differential effects of low and high doses of apelin through opioid receptors on the blood pressure of rats with renovascular hypertension. Hypertension Research. 2017; 40(8):732-737.
Zhou N, Fan X, Mukhtar M, et al. Cell—cell fusion and internalization of the CNS-based, 2003; 307:22-36.

1-17. (canceled)

18. Apelin conjugated to a chelator and labeled with a radioactive element wherein the Apelin amino acid sequence:
a) comprises SEQ ID NO: 1, SEQ ID NO: 2, SEQ ID NO: 3, or SEQ ID NO: 4; or
b) is selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

19. The conjugated and labeled Apelin according to claim 18, wherein the chelator is selected from 6-amino-6 methylperhydro-1,4-diazepinetetraacetic acid (AAZTA), 1,4,7-triazacyclononane-1,4-diacetic acid (NODA), 1,4,7-triazacyclononane,1-glutaric acid-4,7 acetic acid (NODAGA), 1,4,7,10-tetraazacyclododecane-1,4,7,10-tetraacetic acid (DOTA), 2,2′,2″-(10-(2,6-dioxotetrahydro-2H-pyran-3-yl)-1,4,7,10-tetraazacyclododecane-1,4,7-triyl)triacetic acid (DOTAGA), 1,4,7-triazacyclononane-triacetic acid (NOTA), N,N′-Bis(2-hydroxybenzyl)-1-(4-bromoacetamidobenzyl)-1,2-ethylenediamine-N,N′-diacetic acid (HBED), N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide (DFO), triazacyclononane-phosphinate (TRAP), pentetic acid or diethylenetriaminepentaacetic acid (DTPA), bromoacetamidobenzyl(TETA),1,4,7-triazacyclononane-1,4-bis[methylene(hydroxymethyl)phosphinicacid]-7-[methylene(2-carboxyethyl)phosphinicacid])(NOPO), HBED-CC(DKFZ), 2-(4-isothiocyanotobenzyl)-1, 4, 7, 10-tetraaza-1, 4, 7, 10-tetra-(2-carbamonyl methyl)-cyclododecane (TCMC), N—[(R)-2-amino-3-(p-aminophenyl)propyl]-trans-(S,S)-cyclohexane-1,2-diamine-N,N,N′,N″,N″-pentaacetic acid (CHX-A″-DTPA) and a functional derivative thereof.

20. The conjugated and labeled Apelin according to claim 18, wherein the radioactive element is a radionuclide selected from the group consisting of gallium-68 (⁶⁸Ga), gallium-67 (⁶⁷Ga), lutetium-177 (¹⁷⁷Lu), fluorine-18 (F¹⁸), yttrium-90 (⁹⁰Y), bismuth-213 (²¹³Bi), actinium-225 (²²⁵Ac), lead-212 (²¹²Pb), indium-111 (¹¹¹In), zirconium-89 (⁸⁹Zr), terbium-149 (¹⁴⁹Tb), terbium-152 (¹⁵²Tb), terbium-155 (¹⁵⁵Tb), terbium-161 (¹⁶¹Tb) and copper-64 (⁶⁴Cu).

21. The conjugated and labeled Apelin according to claim 18 which is selected from the group consisting of [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, Al[¹⁸F]F-DOTAGA-Apelin, [⁶⁴Cu]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [¹⁷⁷Lu]Lu-DOTA-Apelin, [¹⁷⁷Lu]Lu-DOTAGA-Apelin, [¹⁷⁷Lu]Lu-DKFZ-Apelin, [¹⁷⁷Lu]Lu-AAZTA-Apelin, [²²⁵Ac]Ac-DOTA-Apelin, [Pb²¹²]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin and [¹¹¹In]In-DTPA-Apelin, [¹⁴⁹Tb]Tb-DOTA-Apelin, [¹⁴⁹Tb]Tb-DOTAGA-Apelin, [¹⁵²Tb]Tb-DOTA-Apelin, [¹⁵²Tb]Tb-DOTAGA-Apelin, [¹⁵⁵Tb]Tb-DOTA-Apelin, [¹⁵⁵Tb]Tb-DOTAGA-Apelin, [¹¹⁶Tb]Tb-DOTA-Apelin and [¹¹⁶Tb]Tb-DOTAGA-Apelin.

22. The conjugated and labeled Apelin according to claim 18 which is selected from the group consisting of [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, [¹¹¹In]In-DTPA-Apelin, [Cu⁶⁴]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [¹⁵²Tb]Tb-DOTA-Apelin, [¹⁵²Tb]Tb-DOTAGA-Apelin, [¹⁵⁵Tb]Tb-DOTA-Apelin and [⁵⁵Tb]Tb-DOTAGA-Apelin.

23. The conjugated and labeled Apelin according to claim 18 which is selected from the group consisting of [¹⁷⁷Lu]Lu-DOTA-Apelin, [¹⁷⁷Lu]Lu-DOTAGA-Apelin, [¹⁷⁷Lu]Lu-DKFZ-Apelin, [¹⁷⁷Lu]Lu-AAZTA-Apelin, [²²⁵Ac]Ac-DOTA-Apelin, [Pb²12]Pb-TCMC-Apelin, [²¹³Bi]Bi-DTPA-Apelin, [⁹⁰Y]Y-DTPA-Apelin, [⁹⁰Y]Y-CHX-A″-DTPA-Apelin, [¹⁴⁹Tb]Tb-DOTA-Apelin, [¹⁴⁹Tb]Tb-DOTAGA-Apelin, [¹⁶¹Tb]Tb-DOTA-Apelin, and [¹⁶¹Tb]Tb-DOTAGA-Apelin.

24. A method of labelling or imaging angiogenesis or vasculogenesis in a subject, or of labelling or imaging in vivo or ex vivo a tissue or organ expressing the APJ receptor, comprising administering the subject with, or exposing the tissue or organ to a conjugated and labeled Apelin according to claim 22 used as a Single Photon Emission computed Tomography (SPECT-CT) radiotracer or as a Positron Emission Tomography-Computed Tomography (PET-CT) radiotracer.

25. The method according to claim 24, wherein the subject is a human being.

26. A method for treating angiogenesis, vasculogenesis or a disease or disorder inducing or modulating the expression of an APJ receptor in a tissue or organ in a subject in need thereof, comprising administering the subject with a conjugated and labeled Apelin according to claim 23.

27. The method according to claim 26, wherein the disease or disorder inducing or modulating the expression of an APJ receptor in a tissue or organ is a solid cancer wherein the cancerous tumor and/or cancerous tumor vasculature expresses an APJ receptor, and the cancer is typically selected from lung cancer, cholangiocarcinoma, liver cancer, gastric cancer, prostate cancer, ovarian cancer, breast cancer, renal cancer, squamous cell carcinoma, multiple myeloma, glioblastoma, colon cancer, obesity-related colon cancer, endometrial cancer and obesity-related endometrial cancer.

28. The method according to claim 26, wherein the subject is a human being.

29. A composition comprising an Apelin conjugated to a chelator and labeled with a radioactive element according to claim 18 and a pharmaceutically acceptable diluent, excipient, carrier or support.

30. A method of detecting, measuring, diagnosing, staging or monitoring angiogenesis or vasculogenesis, or a disease or disorder inducing or modulating the expression of an APJ receptor in a tissue or organ in a subject, comprising administering the subject with a conjugated and labeled Apelin selected from [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, [¹¹¹In]In-DTPA-Apelin, [Cu⁶⁴]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [¹⁵²Tb]Tb-DOTA-Apelin, [¹⁵²Tb]Tb-DOTAGA-Apelin, [¹¹⁵Tb]Tb-DOTA-Apelin and [¹⁵⁵Tb]Tb-DOTAGA-Apelin, or with a composition comprising an Apelin conjugated to a chelator and labeled with a radioactive element according to claim 18, wherein the Apelin amino acid sequence comprises SEQ ID NO: 1, SEQ ID NO: 2, SEQ ID NO: 3, or SEQ ID NO: 4, and a pharmaceutically acceptable diluent, excipient, carrier or support.

31. The method according to claim 30, wherein the disease or disorder is selected from ischemia, an ischemia-associated disease or disorder, myocardial infarction, a solid cancer, atherosclerosis, an endothelial dysfunction-related disease, a cardiovascular disease, a metabolic disease, diabetes mellitus and obesity.

32. The method according to claim 30, wherein the subject is a human being.

33. A method of evaluating or monitoring the therapeutic effect of an angiogenic or anti-angiogenic treatment, or of an APJ receptor-targeted treatment, in a subject, comprising administering the subject with a conjugated and labeled Apelin selected from [⁶⁸Ga]Ga-NODAGA-Apelin, [⁶⁸Ga]Ga-DOTA-Apelin, [⁶⁸Ga]Ga-DOTAGA-Apelin, [⁶⁸Ga]Ga-NOTA-Apelin, [⁶⁸Ga]Ga-HBED-Apelin, [⁶⁸Ga]Ga-DFO-Apelin, [⁶⁸Ga]Ga-AAZTA-Apelin, [⁶⁷Ga]Ga-NODAGA-Apelin, [⁶⁷Ga]Ga-DOTA-Apelin, [⁶⁷Ga]Ga-DOTAGA-Apelin, [⁶⁷Ga]Ga-NOTA-Apelin, [⁶⁷Ga]Ga-HBED-Apelin, [⁶⁷Ga]Ga-DFO-Apelin, [⁶⁷Ga]Ga-AAZTA-Apelin, Al[¹⁸F]F-NOTA-Apelin, Al[¹⁸F]F-NODA-Apelin, [¹¹¹In]In-DTPA-Apelin, [Cu⁶⁴]Cu-DOTA-Apelin, [⁶⁴Cu]Cu-DOTAGA-Apelin, [⁸⁹Zr]Zr-DOTA-Apelin, [⁸⁹Zr]Zr-DOTAGA-Apelin, [¹⁵²Tb]Tb-DOTA-Apelin, [¹⁵²Tb]Tb-DOTAGA-Apelin, [¹⁵⁵Tb]Tb-DOTA-Apelin and [¹⁵⁵Tb]Tb-DOTAGA-Apelin, or a composition comprising an Apelin conjugated to a chelator and labeled with a radioactive element according to claim 18, wherein the Apelin amino acid sequence comprises SEQ ID NO: 1, SEQ ID NO: 2, SEQ ID NO: 3, or SEQ ID NO: 4, and a pharmaceutically acceptable diluent, excipient, carrier or support.

34. The method according to claim 33, wherein the disease or disorder is selected from ischemia, an ischemia-associated disease or disorder, myocardial infarction, a solid cancer, atherosclerosis, an endothelial dysfunction-related disease, a cardiovascular disease, a metabolic disease, diabetes mellitus and obesity.

35. The method according to claim 33, wherein the subject is a human being.

36. A kit comprising an Apelin comprising SEQ ID NO: 1, SEQ ID NO: 2, SEQ ID NO: 3, or SEQ ID NO: 4, a chelator selected from 6-amino-6 methylperhydro-1,4-diazepinetetraacetic acid (AAZTA), 1,4,7-triazacyclononane-1,4-diacetic acid (NODA), 1,4,7-triazacyclononane,1-glutaric acid-4,7 acetic acid (NODAGA), 1,4,7,10-tetraazacyclododecane-1,4,7,10-tetraacetic acid (DOTA), 2,2′,2″-(10-(2,6-dioxotetrahydro-2H-pyran-3-yl)-1,4,7,10-tetraazacyclododecane-1,4,7-triyl)triacetic acid (DOTAGA), 1,4,7-triazacyclononane-triacetic acid (NOTA), N,N′-Bis(2-hydroxybenzyl)-1-(4-bromoacetamidobenzyl)-1,2-ethylenediamine-N,N′-diacetic acid (HBED), N1-hydroxy-N1-(5-(4-(hydroxy(5-(3-(4-isothiocyanatophenyl)thioureido)pentyl)amino)-4-oxobutanamido)pentyl)-N4-(5-(N-hydroxyacetamido)pentyl)succinamide (DFO), triazacyclononane-phosphinate (TRAP), pentetic acid or diethylenetriaminepentaacetic acid (DTPA), bromoacetamidobenzyl(TETA),1,4,7-triazacyclononane-1,4-bis[methylene(hydroxymethyl)phosphinicacid]-7-[methylene(2-carboxyethyl)phosphinicacid])(NOPO), HBED-CC(DKFZ), 2-(4-isothiocyanotobenzyl)-1, 4, 7, 10-tetraaza-1, 4, 7, 10-tetra-(2-carbamonyl methyl)-cyclododecane (TCMC), N—[(R)-2-amino-3-(p-aminophenyl)propyl]-trans-(S,S)-cyclohexane-1,2-diamine-N,N,N′,N″,N″-pentaacetic acid (CHX-A″-DTPA) and a functional derivative thereof, and a radioactive element which is a radionuclide selected from gallium-68 (⁶⁸Ga), gallium-67 (⁶⁷Ga), lutetium-177 (¹⁷⁷Lu), fluorine-18 (F¹⁸), yttrium-90 (⁹⁰Y), bismuth-213 (²¹³Bi), actinium-225 (²²⁵Ac), lead-212 (²¹²Pb), indium-111 (¹¹¹In), zirconium-89 (⁸⁹Zr), terbium-149 (¹⁴⁹Tb), terbium-152 (¹⁵²Tb), terbium-155 (⁵⁵Tb), terbium-161 (¹⁶¹Tb) and copper-64 (⁶⁴Cu), in three distinct containers; or an Apelin-chelator conjugate wherein the Apelin amino acid sequence comprises SEQ ID NO: 1, SEQ ID NO: 2, SEQ ID NO: 3, or SEQ ID NO: 4, in a single container, and a radioactive element which is a radionuclide selected from gallium-68 (⁶⁸Ga), gallium-67 (⁶⁷Ga), lutetium-177 (¹⁷⁷Lu), fluorine-18 (F¹⁸), yttrium-90 (⁹⁰Y), bismuth-213 (²¹³Bi), actinium-225 (²²⁵Ac), lead-212 (²¹²Pb), indium-111 (¹¹¹In), zirconium-89 (⁸⁹Zr), terbium-149 (¹⁴⁹Tb) terbium-152 (¹⁵²Tb), terbium-155 (¹⁵⁵Tb) terbium-161 (¹⁶¹Tb) and copper-64 (⁶⁴Cu), in a distinct container.

37. A method for producing an Apelin conjugated to a chelator and labeled with a radioactive element wherein the Apelin amino acid sequence comprises SEQ ID NO: 1, SEQ ID NO: 2, SEQ ID NO: 3, or SEQ ID NO: 4, with the kit of claim 36.
